(19) United States
(12) Patent Application Publication
(10) Pub. No.: US 2026/0315396 A1
(43) Pub. Date: Oct. 15, 2026

(54) DOSAGE FORMS FOR GASTROINTESTINAL SYNTHETIC EPITHELIAL LININGS

(71) Applicant: Syntis Bio, Inc., Boston, MA (US)

(72) Inventors: Mary Ashley Meagan HUDSON, Boston, MA (US); Vasu Venkat SETHURAMAN, Bedford, MA (US)

(21) Appl. No.: 19/642,007

(22) Filed: Apr. 8, 2026

Related U.S. Application Data
(60) Provisional application No. 63/786,131, filed on Apr. 9, 2025.

(51) Int. Cl. A61K 9/20; A61K 9/00

(52) U.S. Cl. CPC A61K 9/2095; A61K 9/0065; A61K 9/2009; A61K 9/2013; A61K 9/2031; A61K 9/2059

(57) ABSTRACT

The disclosure relates to solid dosage forms, such as compressed tablets, for administration to subjects to form synthetic linings for the gastrointestinal (GI) tract, “Gastrointestinal Synthetic Epithelial Linings” or “GSELs.” A GSEL is a temporary lining on the inner surface of the lumen of the GI tract, which can modulate absorption of drugs, nutrients, and other substances in the GI tract. The solid dosage forms disclosed herein provide an efficient and convenient way of delivering the precursor materials for the GSEL to the gastrointestinal tract of a subject.

### CROSS-REFERENCE TO RELATED APPLICATIONS

This application claims priority benefit of U.S. Provisional Patent Application No. 63/786,131, filed Apr. 9, 2025. The contents of that application are hereby incorporated herein by reference in their entirety.

### FIELD OF THE INVENTION

The invention relates to dosage forms for administering synthetic linings for the gastrointestinal (GI) tract, which can modulate absorption of drugs, nutrients, and other substances in the GI tract.

### BACKGROUND OF THE INVENTION

The gastrointestinal (GI) tract in humans, vertebrates, and other animals serves as a highly versatile organ system with multiple functions. A primary function of the GI tract is absorption of substances, such as nutrients from food. Absorption of substances in the GI tract is also widely utilized for administration of pharmaceuticals. Gastrointestinal synthetic epithelial linings (GSELs) are a method of modulating absorption of substances in the GI tract in a subject, for example, blocking uptake of glucose, or enhancing uptake of pharmaceuticals.

Diverse modes are available for administration of the lining to form a layer or a barrier at a target site, such as the gastrointestinal tract. Depending on the formulation, the efficacy for each of these administration routes is variable. For example, some methods such as oral gavage result in material loss before the formulation reaches the desired location. Liquid formulations may require high volumes and/or frequent doses to be ingested or administered to effectively form a barrier or layer at the target site.

There is thus a need for improved delivery forms for GSEL precursors, in order to form GSELs efficiently in the gastrointestinal tract in a manner convenient for the subject.

### SUMMARY OF THE INVENTION

The present disclosure relates to solid dosage forms, such as compressed tablets, for administering precursors of synthetic linings to be formed in situ in the gastrointestinal tract, such as in the small intestine. The synthetic lining can be used per se for treating a disease or disorder, or can be used in combination with an agent such as an active pharmaceutical ingredient. The synthetic lining itself is referred to as a Gastrointestinal Synthetic Epithelial Lining (GSEL), while the GSEL precursor composition is referred to as SYNT.

Disclosed herein is, inter alia, a compressed solid tablet for oral administration for forming a polymer in situ in a subject, comprising a polymer precursor; an oxygen source; and a disintegrant. The maximum dimension of the tablet (i.e., the larger of its length, width, or height) can be about 20 mm or less. The volume of the tablet can be about 1.5 cubic centimeters or less. The polymer precursor can comprise polydopamine, or can comprise polydopamine and dopamine monomers. The oxygen source can comprise a peroxide. The oxygen source can comprise urea hydrogen peroxide, hydrogen peroxide, or calcium peroxide. The disintegrant can be selected from the group consisting of croscarmellose sodium, crospovidone, and sodium starch glycolate. In an embodiment, the disintegrant comprises sodium starch glycolate.

Also disclosed herein is a method of forming a polymer in a gastrointestinal tract of a subject, comprising orally administering one or more compressed solid tablets as disclosed herein, wherein the polymer precursor and the oxygen source contact a catalyst endogenous to the subject and the catalyst polymerizes the polymer precursor to form the polymer.

Also disclosed herein is a method of formulating the compressed solid tablet as disclosed herein, wherein the compressed solid tablet is formulated by combining a polymer precursor, an oxygen source, and a disintegrant in a die, and applying a force to the combined polymer precursor, oxygen source, and disintegrant mixture. The force can be, for example, at least about 12 megapascal, such as a force between 12 megapascal and 36 megapascal.

### BRIEF DESCRIPTION OF THE DRAWINGS

FIG. 1 shows the change in blood glucose levels after SYNT capsule or suspension administration.

FIG. 2A shows the effect of tablet compression and filler type and ratio on tablet tensile strength.

FIG. 2B shows an exemplary SYNT capsule and an exemplary SYNT tablet.

FIG. 3A shows the relationship between tablet compression and tablet size.

FIG. 3B shows exemplary compressed SYNT tablets made with the F21 formulation, with a total tablet weight of 1500 mg and delivering 1102 mg of polymer precursor, oxygen source, and buffer, and exemplary capsules delivering 450 mg of SYNT.

FIG. 4A shows a schematic of the barrier function experiments.

FIG. 4B shows the percent blockade achieved by SYNT tablets compressed at 2000 PSI and 5000 PSI.

FIG. 5 shows the impact of sieving PDA size through various meshes.

FIG. 6A shows images of porcine tissue exposed to SYNT capsules or tablets formulated with or without disintegrant.

FIG. 6B shows images of porcine tissue exposed to SYNT tablets formulated with or without disintegrant and compressed at either 2500 or 5000 PSI.

FIG. 7A shows exemplary SYNT tablets that are not suitable for administration.

FIG. 7B shows the relationship between SYNT formulations and hardness of the resulting tablet.

FIG. 7C shows the excipient ratio of each SYNT formulation as shown in FIG. 8B.

FIG. 7D shows images of exemplary SYNT formulation tablets with various fillers at different ratios.

FIG. 8A shows the results of intraduodenal glucose challenge after administration of SYNT capsule or tablet.

FIG. 8B shows the change in area under curve (AUC) in blood glucose levels after an intraduodenal glucose challenge in pigs who were administered either a SYNT capsule or a SYNT tablet.

FIG. 9 shows an exemplary SYNT compressed tablet formulation.

FIG. 10A shows an overview of the mechanism of the SYNT platform and transient small intestinal coating mechanism.

FIG. 10B shows the comparative catalase expression levels across species.

FIG. 11A shows the change in blood glucose levels after SYNT-101 treatment via suspension form or tablet form in Yorkshire pigs.

FIG. 11B shows the pharmacokinetics of SYNT-101 treatment via suspension form or tablet form in Yorkshire pigs.

FIG. 12A shows the change in blood glucose levels after SYNT-101 treatment in Sprague-Dawley rats.

FIG. 12B shows the changes in body weight in Sprague-Dawley rats over a six-week period after SYNT-101 treatment.

FIG. 12C shows the Echo MRI-based body composition analysis of fat and lean muscle in animals after SYNT-101 treatment.

FIG. 12D shows the changes in leptin and ghrelin levels in animals after SYNT-101 treatment, compared to subcutaneous dosing of semaglutide (SEMA).

FIG. 13A shows the dissolution profiles of enterically-coated tablets.

FIG. 13B shows Pill-Cam imaging of the stomach, small intestine, and colon two-hours post-SYNT administration.

FIG. 13C shows the change in blood glucose levels after SYNT-101 treatment in canines.

FIG. 13D shows the modulation of satiety-related hormone levels during oral glucose tolerance test (OGTT) following SYNT-101 administration.

FIG. 13E shows the modulation of satiety-related hormone levels following SYNT-101 administration.

FIG. 14A shows the change in blood glucose levels in canines after repeat-dosing of SYNT-101.

FIG. 14B shows the IDEXX blood chemistry and safety assessment in canines following repeat-dosing of SYNT-101.

FIG. 14C shows the changes in liver chemistry parameters following SYNT-101 treatment for 28 days.

FIG. 14D shows the changes in levels of cholesterol, triglycerides, high-density lipoprotein (HDL), and low-density lipoprotein (LDL) following SYNT-101 administration for 28 days.

FIG. 15 shows a timeline for the First-in-Human study design.

FIG. 16A shows the change in glucose levels after SYNT-101 treatment in humans.

FIG. 16B shows the changes in metabolic hormone levels leptin and ghrelin in humans three hours post SYNT-101 administration.

FIG. 16C shows the changes in metabolic hormone levels GLP-1 and PYY in humans three hours post SYNT-101 administration.

FIG. 17 shows the safety assessments including clinical observations, day 3 biopsy findings, and liver chemistry parameters (BUN, ALT, AST) in humans post SYNT-101 administration.

FIG. 18 shows a schematic of the Peroral Mucosal Epithelium Absorption Enhancer (PERMEATE) mechanism. PERMEATE leverages the in situ formation of SYNT, which colocalizes and extends the small intestinal residence time of drug molecules and permeation enhancers, allowing for enhanced absorption.

FIG. 19 shows the high throughput permeation screens across pig jejunal tissue, identifying glycocholic acid (GCA) and ammonium carbonate (NHCO) as synergistic permeation enhancers for semaglutide.

FIG. 20A shows a schematic of the colocalization experiments on ex vivo pig duodenal tissue.

FIG. 20B shows the colocalization of FITC-Dextran dyes and SYNT-101 in ex vivo pig duodenal tissue.

FIG. 20C shows the colocalization of semaglutide in ex vivo pig duodenal tissue with and without PERMEATE.

FIG. 20D shows the colocalization of semaglutide in ex vivo pig duodenal tissue with PERMEATE in fasted- and fed-state tissued conditions.

FIG. 21 shows a timeline for in vivo study and calculation of absolute bioavailability in rats and pigs.

FIG. 22A shows the pharmacokinetic (PK) curves of semaglutide in pigs up to 168 hours with and without PERMEATE.

FIG. 22B shows the absolute bioavailabilities of semaglutide in pigs after 168 hours with and without PERMEATE.

FIG. 23A shows the pharmacokinetic (PK) curves of semaglutide in rats up to 24 hours with and without PERMEATE.

FIG. 23B shows the absolute bioavailabilities of semaglutide in rats after 24 hours with and without PERMEATE.

FIG. 23C shows the pharmacokinetic (PK) curves of semaglutide in rats up to 48 hours with and without PERMEATE.

FIG. 23D shows the absolute bioavailabilities of semaglutide in rats after 48 hours with and without PERMEATE.

FIG. 24A shows a flow chart for wet granulation of SYNT.

FIG. 24B shows a flow chart for dry granulation of SYNT.

### DETAILED DESCRIPTION OF THE INVENTION

The current disclosure provides for dosage forms for gastrointestinal synthetic epithelial linings, specifically solid dosage forms or formulations, and in particular compressed tablet dosage forms. The solid dosage forms or formulations, in particular the compressed tablets, decrease the amount and/or frequency of dosages required to assemble a gastrointestinal synthetic epithelial lining in situ, and promote or enhance in situ barrier formation. Solid forms or formulations can reduce the size of the solid dosage form, and can also better control the dissolution and/or delivery of the formulation in situ. The synthetic lining formed in situ in the gastrointestinal tract, such as in the small intestine is called a Gastrointestinal Synthetic Epithelial Lining (GSEL). A GSEL is a temporary polymeric lining that rests against (i.e., contacts) the internal surface of the gastrointestinal tract. For example, dopamine can be polymerized in the small intestine to form a GSEL coating in the interior of the duodenum, jejunum, or ileum. The present disclosure describes the dosage forms and methods of manufacture for making and administering the precursors to form a GSEL. The precursor formulation is referred to as SYNT.

When SYNT is administered to a subject as a tablet or a capsule, variability in delivery to in situ target sites is minimized and more SYNT is available for polymerization at the desired gastrointestinal site, as compared to SYNT liquid formulations. Accordingly, less SYNT precursor composition must be ingested or administered if the SYNT precursor composition is a solid dosage form or formulation compared to a SYNT precursor composition in a liquid dosage form or formulation. Compressing the SYNT precursors into a compressed tablet dosage form increases the amount of SYNT precursors in a single dosage form. This reduces the number of tablets that must be orally administered, providing convenience for the subject. The compressed tablet of SYNT precursors may also provide better formation and coverage of the resulting GSEL lining.

The SYNT can also be formulated and used in combination with an agent and one or more permeation enhancers. The dosage forms described herein can decrease the number of doses of SYNT which must be ingested to form the lining, and can increase the blockade function of the SYNT coating.

GSELS can be used to localize an active pharmaceutical ingredient (API) in a specific location, resulting in prolonged contact with the gastrointestinal mucosa. Due to the prolonged contact with the mucosa, the API can be delivered over an extended period, which can be advantageous for APIs that are absorbed slowly, or which are best administered over a lengthy time period. A solid dosage form results in more concentrated GSEL formation at the optimal location allows for better entrapment of APIs where blockade is most critical, where enzymes can have the greatest impact on toxic metabolites, and where localization of APIs for a prolonged period where permeability is optimal. Controlling the deposit of a GSEL in situ at the optimal locations of the intestine is an important feature, because less formation at the optimal location can result in GSEL inefficiency. Solid dosage forms or formulations of SYNT can permit a more concentrated delivery of SYNT at the optimal location of the intestine, allowing GSEL formation with administration of a lower dosage of SYNT than would be required with liquid formulations. Solid dosage forms or formulations of SYNT can also reduce the frequency of administration by providing sufficient amounts of SYNT to the gastrointestinal tract, as compared to multiple administrations of a liquid dose formulation over a period of time.

In order to form a GSEL, the SYNT precursor composition of the GSEL is administered to a subject orally, such as in powder form. The powder can be delivered in a capsule. Alternatively, the SYNT powder can be compressed into a tablet, and administered as a compressed tablet. The SYNT composition for administration comprises a polymer precursor (e.g., a monomer, oligomer, or other prepolymer, such as polydopamine, dopamine, dopamine oligomers, or dopamine prepolymers). This polymer precursor is polymerized to form the GSEL. The SYNT composition for administration also comprises an oxygen source, alternatively described as an oxygen precursor, such as a peroxide. The SYNT composition for administration also comprises an oxygen source, alternatively described as an oxygen precursor (such as calcium peroxide, hydrogen peroxide or urea hydrogen peroxide), which will provide oxygen at the appropriate location in the gastrointestinal tract to cause polymerization of the polymer precursor. Additional oxygen sources that can be used in SYNT include sodium percarbonate or hydrogen peroxide-polyvinylpyrrolidone complexes (such as Peroxydone™, sold by Ashland Inc., Wilmington, Delaware, USA). The composition relies on one or more catalysts endogenous to the gastrointestinal tract to convert the oxygen precursor (oxygen source) to oxygen. Liberation of oxygen in turn leads to polymerization of the polymer precursor. The small intestine contains endogenous catalysts with peroxidase activity such as catalase, which enables liberation of oxygen, resulting in formation and deposition of a GSEL in the specific anatomical location where the endogenous catalyst is located. A compressed tablet SYNT precursor composition can also include a disintegrant, so that the tablet disintegrates rapidly at the desired site of GSEL formation.

When used with an agent, such as an active pharmaceutical ingredient, the SYNT composition for administration may also comprise one or more permeation enhancers, such as those described in WO2024/155958 and WO2025/072841, which are incorporated herein by reference in their entirety, to increase the permeation of the active pharmaceutical ingredient (API) across the gastrointestinal barrier. Formation of a GSEL with an associated API localizes the API to the vicinity of the polymer. In this manner, a depot of the API and the one or more permeation enhancers can be retained at a specific anatomical location over an extended period of time for absorption at that location. For example, when a GSEL containing API and one or more permeation enhancers forms in the small intestine, the GSEL keeps the API in proximity to the intestinal epithelium for an extended period.

The components of the solid dosage form or formulation of SYNT are discussed in more detail herein, as follows: 1) polymer precursors such as dopamine and polydopamine, 2) oxygen sources such as peroxides, 3) excipients, such as disintegrants, fillers, lubricants, and glidants, and 4) permeation enhancers. SYNT compositions for administration may also contain 5) one or more active pharmaceutical ingredients (APIs). The SYNT solid dosage form or formulations as described herein may also be subject to high pressure compression to form compressed tablets.

### Definitions

An “agent” is any substance intended for therapeutic, diagnostic, or nutritional use in a patient, individual, or subject. Agents include, but are not limited to, drugs, nutrients, vitamins, and minerals. An “active pharmaceutical ingredient” (API) is an agent which is the component in a pharmaceutical dosage that produces the desired biological effect on a patient, individual, or subject to whom the dosage is administered.

An “excipient” is any substance added to a formulation of an agent that is not the agent itself. Excipients include, but are not limited to, binders, coatings, diluents, disintegrants, emulsifiers, flavorings, glidants, lubricants, and preservatives.

A “patient,” “individual,” or “subject” refers to a human, or a non-human animal. Non-human animals include mammals, including domestic animals such as a dog or cat; or an animal used in commerce, such as a cow, pig, horse, sheep, or goat. In a preferred embodiment, a patient, individual, or subject is a human.

“Treating” a disease or disorder with the compositions and methods disclosed herein is defined as administering one or more of the compositions disclosed herein to a patient in need thereof, with or without additional agents, in order to reduce or eliminate either the disease or disorder, or one or more symptoms of the disease or disorder, or to suppress symptoms of the disease or disorder, or to retard the progression of the disease or disorder or of one or more symptoms of the disease or disorder, or to reduce the severity of the disease or disorder or of one or more symptoms of the disease or disorder. Treatment can commence after emergence of symptoms of a disease or disorder, or can commence before emergence of symptoms of a disease or disorder. Treatment can also be continued after symptoms have been resolved.

“Therapeutic use” of the compositions disclosed herein is defined as using one or more of the compositions disclosed herein to treat a disease or disorder, as defined above. A “therapeutically effective amount” of a therapeutic agent, such as a drug, is an amount of the agent, which, when administered to a patient, individual, or subject, is sufficient to achieve the desired biological result, such as an amount sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient, individual, or subject as a single dose, or can be divided and administered as multiple doses.

“Permeation” of an agent or of an active pharmaceutical ingredient (API) across a barrier refers to the amount of the agent or API able to cross the barrier.

A “permeation enhancer” refers to a substance which is able to increase the permeation of an agent or an active pharmaceutical ingredient across a barrier.

The term “polymer” refers to a compound comprising eleven or more covalently connected repeating units.

The term “oligomer” refers to a compound comprising between two to ten covalently connected repeating units.

The unit “kD” refers to kilodaltons.

As used herein, the singular forms “a”, “an”, and “the” include plural references unless indicated otherwise or the context clearly dictates otherwise.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present disclosure.

### Solid Compositions for Administration of SYNT/Gastrointestinal Synthetic Epithelial Linings (GSELs)

The SYNT compositions for formation of the GSEL comprise 1) a polymer precursor; and 2) an oxygen source. In one embodiment, each component of the composition can be administered as a separate dosage form, to combine in the gastrointestinal tract of a subject. In one embodiment, the polymer precursor and oxygen source can be administered together in one dosage form. The SYNT composition can also comprise 3) one or more excipients, such as a disintegrant. In one embodiment, the polymer precursor, the oxygen source, and the one or more excipients can be administered together in one dosage form, such as a compressed tablet or a capsule.

In one embodiment, the SYNT composition can comprise 4) one or more permeation enhancers. The one or more permeation enhancers can be administered in a separate dosage form. In one embodiment, the polymer precursor, oxygen source, one or more excipients, and one or more permeation enhancers can be administered together in one dosage form.

The SYNT composition (GSEL precursor composition) can also comprise 5) an agent, such as an active pharmaceutical ingredient (API); or the SYNT composition can comprise multiple agents such as multiple APIs. As with the polymer precursor, oxygen source, excipient(s), and one or more permeation enhancers, the agent can be administered together with the other components of the SYNT composition. Alternatively, an API (or multiple APIs) can be administered in a separate composition, either before administration of the other components of the SYNT composition, concurrently with administration of the other components of the SYNT composition, or subsequent to administration of the other components of the SYNT composition.

### Polymer Precursors for Gastrointestinal Synthetic Epithelial Lining Precursor Compositions

In one aspect, the disclosure provides a method of forming a polymer in situ in a subject, the method comprising administering to a subject a composition comprising a polymer precursor, an oxygen source, optionally one or more excipients, and optionally one or more permeation enhancers, wherein the polymer precursor and the oxygen source contact a catalyst endogenous to the subject and the catalyst polymerizes the polymer precursor.

In some embodiments, polymerization of the polymer precursor is catalyzed when the oxygen source and polymer precursor contact the endogenous catalyst. In some embodiments, polymerization of the polymer precursor is catalyzed when the oxygen source and polymer precursor contact an endogenous peroxidase in the small intestine. In some embodiments, polymerization of the polymer precursor is catalyzed when the oxygen source and polymer precursor contact an endogenous peroxidase in the duodenum. In some embodiments, polymerization of the polymer precursor is catalyzed when the oxygen source and polymer precursor contact an endogenous catalase in the small intestine. In some embodiments, polymerization of the polymer precursor is catalyzed when the oxygen source and polymer precursor contact an endogenous catalase in the duodenum.

In some embodiments, the polymer precursor is a catechol-based polymer precursor. In some embodiments, the polymer precursor comprises a 1,2-dihydroxybenzene moiety. In some embodiments, the polymer precursor comprises an optionally substituted 1,2-dihydroxybenzene moiety. In some embodiments, the polymer precursor comprises a 1,2-dihydroxyphenyl moiety. In some embodiments, the polymer precursor comprises an optionally substituted 1,2-dihydroxyphenyl moiety.

In some embodiments, the polymer precursor is a 2-(3,4-dihydroxyphenyl)ethylamine-based polymer precursor. (2-(3,4-dihydroxyphenyl)ethylamine is commonly known as dopamine.) In certain embodiments, the polymer precursor comprises a 3,4-dihydroxyphenethylamine moiety. In certain embodiments, the polymer precursor comprises an optionally substituted 3,4-dihydroxyphenethylamine moiety. In certain embodiments, the polymer precursor comprises 2-(3,4-dihydroxyphenyl)ethylamine. In certain embodiments, the polymer precursor comprises an optionally substituted 2-(3,4-dihydroxyphenyl)ethylamine moiety.

In some embodiments, the polymer precursor is selected from the group consisting of polydopamine, dopamine, levodopa, norepinephrine, methyldopa, levodopa methyl ester, levodopa ethyl ester, derivatives thereof, and combinations thereof. In certain embodiments, the polymer precursor is selected from the group consisting of polydopamine, dopamine, levodopa, norepinephrine, methyldopa, levodopa methyl ester, and levodopa ethyl ester. In certain embodiments, the polymer precursor comprises polydopamine and dopamine.

In certain embodiments, the polymer precursor is a derivative of dopamine. In some embodiments, the polymer precursor comprises dopamine, levodopa, norepinephrine, methyldopa, levodopa methyl ester, or levodopa ethyl ester.

In some embodiments, the derivative of the polymer precursor comprises a macromolecule. In some embodiments, the polymer precursor comprises dopamine, levodopa, norepinephrine, methyldopa, levodopa methyl ester, or levodopa ethyl ester and at least one macromolecule. In certain embodiments the macromolecule is alginate, hyaluronic acid, polyacrylic acid, polyethylene glycol, chondroitin sulfate, chitosan, or a combination thereof. In some embodiments the macromolecule is alginate, hyaluronic acid, polyacrylic acid, polyethylene glycol, chondroitin sulfate, or chitosan. In certain embodiments the polymer precursor comprises (A) dopamine, levodopa, norepinephrine, methyldopa, levodopa methyl ester, or levodopa ethyl ester moieties bound to (B) alginate, hyaluronic acid, polyacrylic acid, polyethylene glycol, chondroitin sulfate, or chitosan. In certain embodiments the polymer precursor comprises (A) more than one of dopamine, levodopa, norepinephrine, methyldopa, levodopa methyl ester, and levodopa ethyl ester moieties bound to (B) alginate, hyaluronic acid, polyacrylic acid, polyethylene glycol, chondroitin sulfate, or chitosan. In certain embodiments the polymer precursor comprises (A) more than one of dopamine, levodopa, norepinephrine, methyldopa, levodopa methyl ester, and levodopa ethyl ester moieties bound to (B) more than one of alginate, hyaluronic acid, polyacrylic acid, polyethylene glycol, chondroitin sulfate, and chitosan.

In some embodiments, the polymer precursor comprises polydopamine. In some embodiments, the polymer precursor comprises polydopamine and dopamine monomers. In some embodiments, the polymer precursor comprises polydopamine, dopamine oligomers, and dopamine monomers. In some embodiments, the polymer precursor comprises dopamine oligomers and dopamine monomers. In some embodiments, the polymer precursor comprises dopamine oligomers. In some embodiments, the polymer precursor comprises dopamine monomers. In some embodiments, the polymer precursor comprises polydopamine and dopamine oligomers.

TABLE 1
Polymer precursors

| Polymer | |
|---|---|
| precursor | Structure |
| M1 | 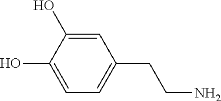 [SMILES: NCCc1ccc(O)c(O)c1] |
| M2 | 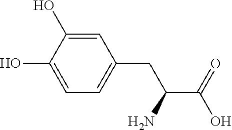 [SMILES: N[C@@H](Cc1ccc(O)c(O)c1)C(=O)O] |
| M3 | 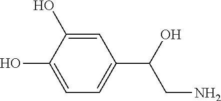 [SMILES: NCC(O)c1ccc(O)c(O)c1] |
| M4 | 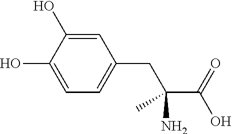 |
| M5 | 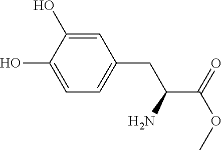 |
| M6 | 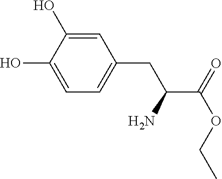 [SMILES: CCOC(=O)[C@@H](N)Cc1ccc(O)c(O)c1] |

TABLE 2
Macromolecules for Use as Polymer Precursors

| Macromolecule | Components | Structure |
|---|---|---|
| C1 | M1 and alginate | 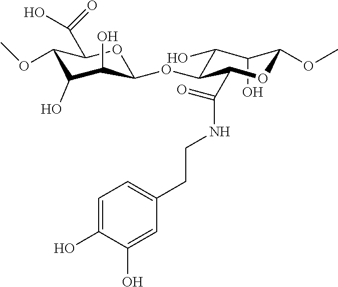 |
| C2 | M2 and alginate | 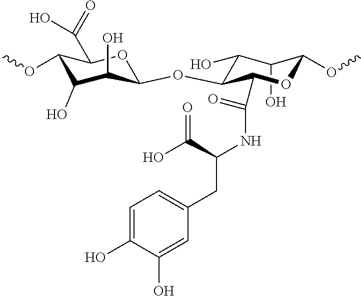 |
| C3 | M3 and alginate | 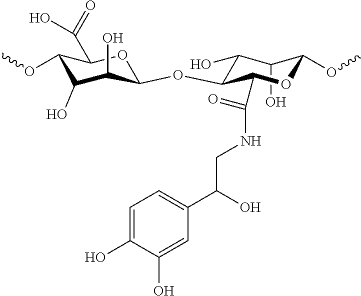 |
| C4 | M4 and alginate | 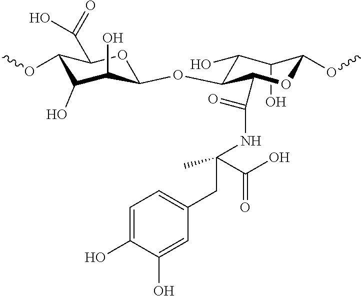 |
| C5 | M5 and alginate | 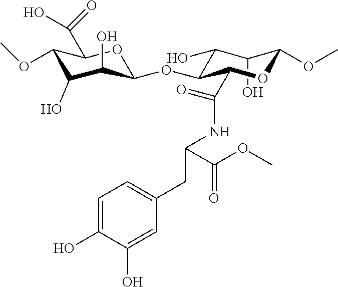 |
| C6 | M6 and alginate | 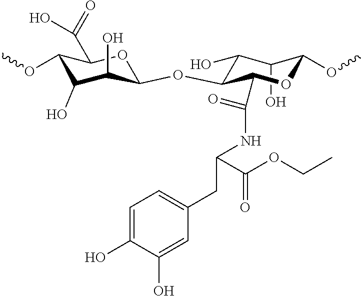 |
| C7 | M1 and hyaluronic acid | 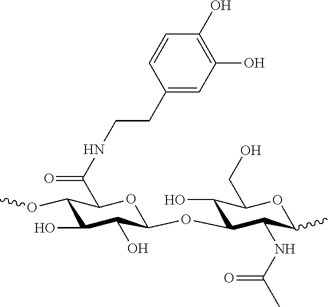 |
| C8 | M2 and hyaluronic acid | 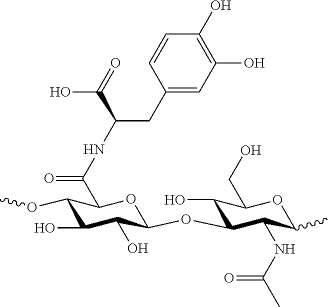 |
| C9 | M3 and hyaluronic acid | 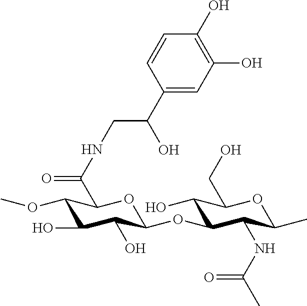 |
| C10 | M4 and hyaluronic acid | 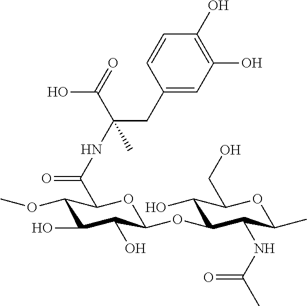 |
| C11 | M5 and hyaluronic acid | 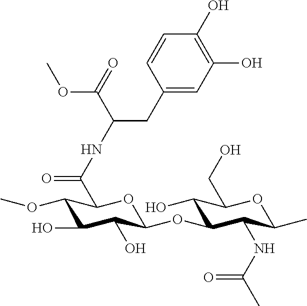 |
| C12 | M6 and hyaluronic acid | 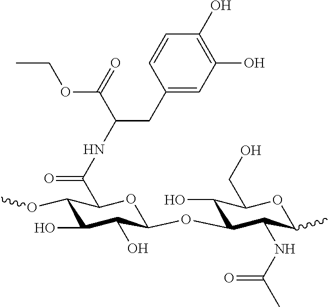 |
| C13 | M1 and polyacrylic acid | 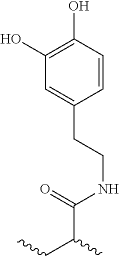 |
| C14 | M2 and polyacrylic acid | 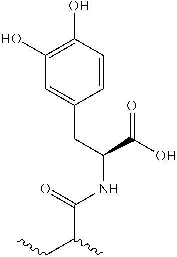 |
| C15 | M3 and polyacrylic acid | 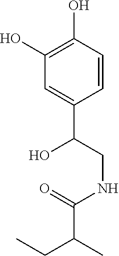 [SMILES: CCC(C)C(=O)NCC(O)c1ccc(O)c(O)c1] |
| C16 | M4 and polyacrylic acid | 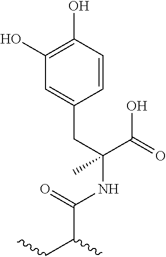 |
| C17 | M5 and polyacrylic acid | 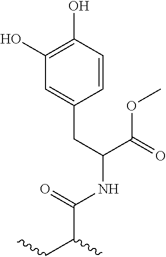 |
| C18 | M6 and polyacrylic acid | 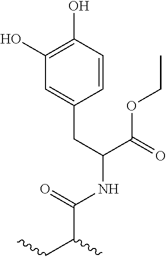 |
| C19 | M1 and polyethylene with tripentaerythritol core | 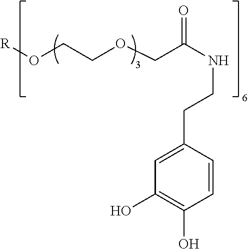 R-tripentaerythritol core structure |
| C20 | M1 and chondroitin sulfate | 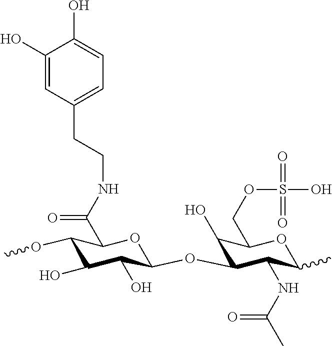 |
| C21 | M1 and chitosan | 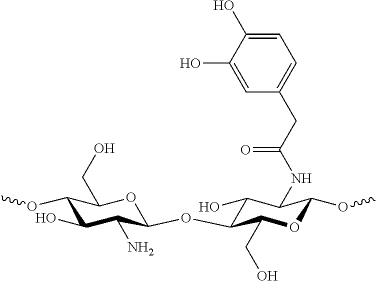 |

In some embodiments, the polymer precursor comprises a single type of polymer precursor. In certain embodiments, the polymer precursor comprises a combination of polymer precursors. In certain embodiments, the polymer precursor comprises a combination of polymer precursors listed in Tables 1 and 2. In some embodiments, the combination of polymer precursors consists of two different polymer precursors. In some embodiments, the combination of polymer precursors consists of three different polymer precursors. In some embodiments, the combination of polymer precursors consists of four different polymer precursors.

International Patent Application Nos. WO 2021/119350 and WO 2021/119354, which are incorporated herein by reference in their entirety, describe methods of forming polymers in situ in a subject.

### Oxygen Sources for Gastrointestinal Synthetic Epithelial Lining Precursor Compositions

In one aspect, the disclosure provides for a composition comprising a polymer precursor, an oxygen source, and a permeation enhancer. In some embodiments, the oxygen source is calcium peroxide, hydrogen peroxide or urea hydrogen peroxide. In some embodiments, the oxygen source is calcium peroxide. In some embodiments, the oxygen source is hydrogen peroxide. In some embodiments, the oxygen source is urea hydrogen peroxide. When the oxygen source contacts a catalyst endogenous to the subject, such as a catalase or a peroxidase, oxygen is liberated from the oxygen source, resulting in polymerization of the polymer precursor.

In some embodiments, the endogenous catalyst is selected from catalases or peroxidases. In some embodiments, the endogenous catalyst is a peroxidase. In certain embodiments, the peroxidase is eosinophil peroxidase, lactoperoxidase, or myeloperoxidase.

In some embodiments, the endogenous catalyst is a catalase. In some embodiments, the catalase is a bacterial catalase. In some embodiments, the catalase is a human catalase.

In some embodiments, the endogenous catalyst is located in the gastrointestinal (GI) tract of the subject. In some embodiments, the endogenous catalyst is located in the small intestine of the subject. In some embodiments, the endogenous catalyst is located in the duodenum of the subject.

In some embodiments, the endogenous catalyst is located in the upper GI of the subject. In some embodiments, the endogenous catalyst is located in the stomach of the subject.

In some embodiments, the composition comprises an amount of oxygen source to (polymer precursor, oxygen source, and permeation enhancer) of 1% to 15%.

### Permeation Enhancers for SYNT/GSEL

The solid formulation may comprise permeation enhancers for inclusion in SYNT and the resulting GSEL, which promote penetration of active pharmaceutical ingredients (APIs) across the gastrointestinal mucosa. A GSEL will localize an API in a specific location, resulting in prolonged contact with the gastrointestinal mucosa. However, some APIs may not permeate through the gastrointestinal barrier in sufficient amounts to be therapeutically effective, or may not permeate at all, despite the prolonged contact afforded by a GSEL lining. Permeation enhancers can be used in conjunction with GSELs to increase the penetration of an API across the gastrointestinal mucosa.

Permeation enhancers may enhance paracellular transport. Paracellular transport is the passive transport of substances or molecules (e.g. active pharmaceutical ingredient) between adjacent epithelial cells. Permeation enhancers that improve paracellular transport may function, for example, by increasing the permeability of tight junctions. In some embodiments, the permeation enhancer enhances paracellular transport. Permeation enhancers may enhance transcellular transport. Transcellular transport is an active process, where a substance or molecule (e.g., an active pharmaceutical ingredient) is transported through a cell using a cellular transport mechanism. In some embodiments, the permeation enhancer enhances transcellular transport. In some embodiments, the permeation enhancer enhances both paracellular and transcellular transport.

In some embodiments, SYNT comprises an amount of one or more permeation enhancers to (polymer precursor, oxygen source, and one or more permeation enhancers) of 0.1% to 60%. In some embodiments, the composition comprises an amount of one or more permeation enhancers to (polymer precursor, oxygen source, and one or more permeation enhancers) of about 0.1% to about 50%, about 0.1% to about 40%, about 0.1% to about 30%, about 0.1% to about 20%, about 0.1% to about 10%, about 1% to about 60%, about 10% to about 60%, about 20% to about 60%, about 30% to about 60%, about 40% to about 60%, or about 50% to about 60%.

Certain combinations of permeation enhancers have been found to work in synergistic combination. In particular, combinations of a) at least one of an ammonium salt, a carbonate salt, or a bicarbonate salt, and b) at least one of a bile salt or bile acid, have been found to have synergistic effects on permeation of agents such as active pharmaceutical ingredients.

The first component of the synergistic permeation enhancer combination comprises one or more of an ammonium salt, a carbonate salt, or a bicarbonate salt. The first component can comprise one or more ammonium salts, one or more carbonate salts, one or more bicarbonate salts, or any number of ammonium salts, any number of carbonate salts, and/or any number of bicarbonate salts in combination.

In some embodiments, the composition comprises ammonium salt. Ammonium salts for use in the synergistic combinations include, but are not limited to, ammonium carbonate (NHCO), ammonium sulfate (NHSO), ammonium citrate (NHCA), ammonium phosphate (NHPO), diammonium phosphate (DAP), monoammonium phosphate (MAP), ammonium bicarbonate (NHHCO), or ammonium chloride (NHCl). Ammonium lactate, ammonium acetate, ammonium sulfate, ammonium sulfite, triammonium citrate, ammonium propionate, and ammonium sulfamate can also be used. One particularly useful ammonium salt is ammonium carbonate. Another particularly useful ammonium salt is ammonium bicarbonate. Abbreviations used herein, such as NHCO to indicate ammonium carbonate, are not intended to designate chemical formulas, as the chemical formula for ammonium carbonate is (NH₄)₂CO₃.

Carbonate salts for use in the synergistic combinations include, but are not limited to, sodium carbonate (NaCO) or potassium carbonate (KCO). As previously noted, these are abbreviations, not chemical formulas.

Bicarbonate salts for use in the invention include, but are not limited to, sodium bicarbonate (NaHCO) or potassium bicarbonate (KHCO). As previously noted, these are abbreviations, not chemical formulas.

The second component of the synergistic permeation enhancer combination comprises one or more of a bile salt or a bile acid. The second component can comprise one or more bile salts, one or more bile acids, or any number of bile salts and/or bile acids in combination. Depending on the pH at which the bile salt or bile acid is prepared, a bile acid may be present as the acid form, as its bile salt form, or as a mixture of the bile acid and its bile salt (i.e., the conjugate base of the bile acid). For example, the pKa of cholic acid is given as 4.98 (see URL pubchem.ncbi.nlm.nih.gov/compound/Cholic-acid, referencing Serjeant EP, Dempsey B; Ionization constants of organic acids in aqueous solution. IUPAC Chem Data Ser No. 23. NY, NY: Pergamon pp. 989 (1979)). Accordingly, when a bile salt or bile acid is recited, it is understood that the form of the material may change depending on the conditions of preparation, especially if preparation includes an aqueous step. That is, a bile salt may convert partially or mostly to a bile acid, and/or a bile acid may convert partially or mostly to a bile salt, depending on the pH of any solution containing the bile salt or bile acid.

Bile acids for use in the synergistic combinations include, but are not limited to, cholic acid (CHA), glycocholic acid (GCA), deoxycholic acid (DCA), glycochenodeoxycholic acid (GCDCA), glycodeoxycholic acid (GDCA), taurodeoxycholic acid (TDCA), taurocholic acid (TCA), bovine bile (OX), chenodeoxycholic acid (CDCA), taurochenodeoxycholic acid (TCDCA), lithocholic acid (LCA), glycolithocholate (GLC), glycohyocholate (GHC), taurolithocholate (TLC), ursodeoxycholic acid (UDCA), tauroursodeoxycholic acid (TUDCA), glycoursodeoxycholic acid (GUDCA), 12-monoketocholic acid (12-MKC), 7-monoketocholic acid (7-MKC), 7,12-diketocholic acid (7,12-DKC), 3,7,12-triketocholic acid (3,7,12-TKC), 12-monoketodeoxycholic acid (12-MKDC), or 3-[(3-cholamidopropyl)dimethylammonio]-1-propanesulfonate (CHAPS). A particularly useful bile acid is glycocholic acid.

Bile salts for use in the synergistic combinations include, but are not limited to, a salt of cholic acid, a salt of glycocholic acid, a salt of deoxycholic acid, a salt of glycochenodeoxycholic acid, a salt of glycodeoxycholic acid, a salt of taurodeoxycholic acid, a salt of taurocholic acid, a salt of bovine bile, a salt of chenodeoxycholic acid, a salt of taurochenodeoxycholic acid, a salt of lithocholic acid, a salt of glycolithocholate, a salt of glycohyocholate, a salt of taurolithocholate, a salt of ursodeoxycholic acid, a salt of tauroursodeoxycholic acid, a salt of glycoursodeoxycholic acid, a salt of 12-monoketocholic acid, a salt of 7-monoketocholic acid, a salt of 7,12-diketocholic acid, a salt of 3,7,12-triketocholic acid, a salt of 12-monoketodeoxycholic acid, or a salt of 3-[(3-cholamidopropyl)dimethylammonio]-1-propanesulfonate. A particularly useful bile salt is a salt of glycocholic acid.

The bile salts can be sodium salts, including, but not limited to, sodium cholate, sodium glycocholate, sodium deoxycholate, sodium glycochenodeoxycholate, sodium glycodeoxycholate, sodium taurodeoxycholate, sodium taurocholate, a sodium salt of bovine bile, sodium chenodeoxycholate, sodium taurochenodeoxycholate, sodium lithocholate, sodium glycolithocholate, sodium glycohyocholate, sodium taurolithocholate, sodium ursodeoxycholate, sodium tauroursodeoxycholate, sodium glycoursodeoxycholate, sodium 12-monoketocholate, sodium 7-monoketocholate, sodium 7,12-diketocholate, sodium 3,7,12-triketocholate, sodium 12-monoketodeoxycholate, or sodium 3-[(3-cholamidopropyl)dimethylammonio]-1-propanesulfonate. A particularly useful sodium salt of a bile acid is sodium glycocholate (GCA). Specific synergistic combinations of permeation enhancers include: glycocholic acid and ammonium carbonate; a salt of glycocholic acid and ammonium carbonate and sodium glycocholate and ammonium carbonate.

A specific synergistic combination of permeation enhancers (expressed as a weight-based dosage) includes: 6.4 mg/kg glycocholic acid (GCA) and 6.4 mg/kg ammonium carbonate (NHCO).

A specific synergistic combination of permeation enhancers is glycocholic acid (GCA) and ammonium carbonate (NHCO).

Where chemically possible and pharmaceutically desirable, alternative pharmaceutically acceptable salts or the free acids or free bases of any of the foregoing compounds can be used.

### Fillers for SYNT

The solid dosage form or formulation may comprise fillers, which acts as a bulking agent. Fillers add substance such that the solid dose form or formulation is made with the correct physical size, and aids in administration of SYNT.

In some embodiments, the composition comprises an amount of a filler of about 20% to about 60% of the total composition. In some embodiments, the composition comprises an amount of a filler of 20% to 60% of the total composition. In some embodiments, the composition comprises an amount of a filler of about 20% to about 50%, about 20% to about 40%, about 20% to about 30%, about 20% to about 60%, about 30% to about 60%, about 40% to about 60%, about 50% to about 60%, about 40% to about 60%, or about 50% to about 60% of the total composition.

In some embodiments, the composition comprises an amount of a filler of about 20% to about 80% of the total composition. In some embodiments, the composition comprises an amount of a filler of 20% to 70% of the total composition. In some embodiments, the composition comprises an amount of a filler of about 20% to about 80%, about 20% to about 70%, about 30% to about 80%, about 30% to about 70%, about 40% to about 80%, about 40% to about 70%, about 50% to about 80%, or about 50% to about 70% of the total composition.

In some embodiments, the composition comprises one or more fillers of a combined amount of about 20% to about 60% of the total composition. In some embodiments, the composition comprises one or more fillers of a combined amount of 20% to 60% of the total composition. In some embodiments, the composition comprises one or more fillers of a combined amount of about 20% to about 50%, about 20% to about 40%, about 20% to about 30%, about 20% to about 60%, about 30% to about 60%, about 40% to about 60%, about 50% to about 60%, about 40% to about 60%, or about 50% to about 60% of the total composition. In some embodiments, the composition comprises one or more fillers of a combined amount of about 20%.

In some embodiments, the composition comprises one or more fillers of a combined amount of about 20% to about 80% of the total composition. In some embodiments, the composition comprises one or more fillers of a combined amount of 20% to 70% of the total composition. In some embodiments, the composition comprises one or more fillers of a combined amount of about 20% to about 80%, about 20% to about 70%, about 30% to about 80%, about 30% to about 70%, about 40% to about 80%, about 40% to about 70%, about 50% to about 80%, or about 50% to about 70% of the total composition.

In some embodiments, the filler is selected from any one of dicalcium phosphate, microcrystalline cellulose, lactose, starch, mannitol, or any combination thereof. In some embodiments, the filler is dicalcium phosphate, microcrystalline cellulose, lactose, starch, and/or mannitol. In some embodiments, the filler is dicalcium phosphate, microcrystalline cellulose, lactose, starch, or mannitol. In some embodiments, the filler is dicalcium phosphate. In some embodiments, the filler is microcrystalline cellulose.

### Glidant for SYNT

The solid formulation may comprise a glidant for SYNT, which reduces powder cohesion and promotes powder flowability.

In some embodiments, the composition comprises an amount of glidant of about 2% to about 10% of the total composition. In some embodiments, the composition comprises an amount of glidant of 0.1% to 2% of the total composition. In some embodiments, the composition comprises an amount of glidant of about 0.1% to about 0.5%, about 0.1% to about 1%, about 0.1% to about 2%, about 0.1% to about 1.5%, about 0.1% to about 0.25%, about 0.2% to about 2%, about 0.3% to about 2%, about 0.4% to about 2%, about 0.75% to about 2%, about 1% to about 2%, about 1.25% to about 2%, about 1.5% to about 2, or about 1.75% to about 2% of the total composition. In some embodiments, the composition comprises an amount of glidant of about 0.5%. In some embodiments, the glidant is colloidal silicon dioxide, talc, or corn starch. In some embodiments, the glidant is colloidal silicon dioxide.

### Disintegrant for SYNT

The solid formulation may comprise disintegrant for SYNT, which promotes the dissolvability of the solid dosage form or formulation. Dissolution of the solid dosage form or formulation of SYNT enhances in situ deposit of the resulting GSEL.

In some embodiments, the composition comprises an amount of disintegrant of about 2% to about 10% of the total composition. In some embodiments, the composition comprises an amount of disintegrant of 2% to 10% of the total composition. In some embodiments, the composition comprises an amount of disintegrant of about 2% to about 10%, about 2% to about 8%, about 2% to about 6%, about 2% to about 4%, about 2% to about 3%, about 4% to about 10%, about 5% to about 10%, about 6% to about 10%, about 7% to about 10%, about 8% to about 10%, or about 9% to about 10% of the total composition. In some embodiments, the composition an amount of disintegrant of about 5%. In some embodiments, the composition an amount of disintegrant of about 5%. In some embodiments, the disintegrant is sodium starch glycolate, croscarmellose sodium, or crospovidone. In some embodiments, the disintegrant is sodium starch glycolate. In some embodiments, the disintegrant is sodium starch glycolate, Type A.

### Binders for SYNT

The solid formulation may comprise binders for SYNT, which promotes granulation and maintenance of shape of the solid SYNT composition.

In some embodiments, the composition comprises an amount of binder of 5% or less. In some embodiments, the composition comprises an amount of binder of about 0% to about 5%, about 0% to about 4%, about 0% to about 3%, about 0% to about 2%, about 0% to about 1%, about 1% to about 5%, about 1% to about 4%, about 1% to about 3%, about 1% to about 2%, about 2% to about 5%, about 2 to about 4%, or about 3% to about 4%. In some embodiments, the binder is polyvinylpyrrolidone, hydroxypropyl cellulose, or hydroxypropyl methylcellulose.

### Lubricant for SYNT

The solid formulation may comprise a lubricant for SYNT, which can be included to counter solid dosage formulation adhesion.

In some embodiments, the composition comprises an amount of lubricant of about 2% to about 10% of the total composition. In some embodiments, the composition comprises an amount of lubricant of 0.25% to 2% of the total composition. In some embodiments, the composition comprises an amount of lubricant of about 0.25% to about 0.5%, about 0.25% to about 1%, about 0.25% to about 2%, about 0.25% to about 1.5%, about 0.25% to about 1.75%, about 0.3% to about 2%, about 0.4% to about 2%, about 0.75% to about 2%, about 1% to about 2%, about 1.25% to about 2%, about 1.5% to about 2, or about 1.75% to about 2% of the total composition. In some embodiments, the composition comprises an amount of lubricant of about 1.0%. In some embodiments, the lubricant is magnesium stearate, stearic acid, or sodium stearyl fumarate. In some embodiments, the lubricant is magnesium stearate.

### Buffers for SYNT

The solid formulation may comprise buffers, such as Tris base, Tris acid, or a combination of Tris base and Tris acid.

### Exemplary Formulations

An exemplary SYNT formulation, with lower and upper bounds on the various components, is provided in the table below.

| | | | Lower | Upper |
|---|---|---|---|---|
| | | | bound (mg) | bound (mg) |
| | Lower | Upper | for 1000 | for 1000 |
| Component | bound | bound | mg tablet | mg tablet |
| PDA | 20% | 60% | 200 | 600 |
| Dopamine | 10% | 30% | 100 | 300 |
| Tris base | 5% | 10% | 50 | 100 |
| Tris acid | 4% | 6% | 40 | 60 |
| Calcium peroxide, | 1% | 3% | 10 | 30 |
| Urea Peroxide | | | | |
| Filler (Dicalcium | 20% | 60% | 200 | 600 |
| phosphate, | | | | |
| Microcrystalline | | | | |
| cellulose, Lactose, | | | | |
| Starch, Mannitol) | | | | |
| Disintegrant (Sodium | 2% | 10% | 20 | 100 |
| starch glycolate, | | | | |
| Croscarmellose | | | | |
| Sodium, Crospovidone) | | | | |
| Lubricant (Magnesium | 0.25% | 2% | 2.5 | 20 |
| stearate, stearic | | | | |
| acid, sodium | | | | |
| stearyl fumarate) | | | | |
| Glidant (Colloidal | 0.1% | 2% | 1 | 20 |
| silicon dioxide, talc, | | | | |
| corn starch) | | | | |

### Wet Granulation

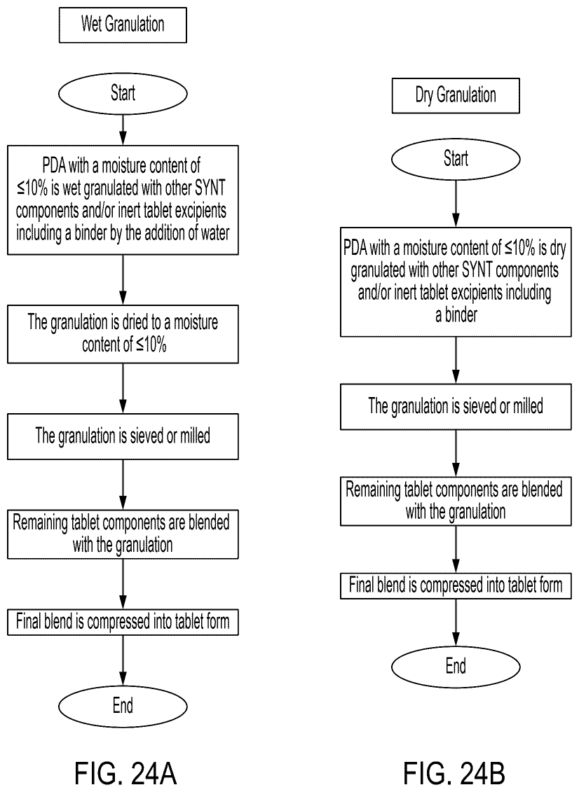

A controlled wet granulation step can be used to avoid the need for tight control of the polydopamine (PDA) moisture content as granulation can be a method to improve tablet robustness. FIG. 24A outlines the basic steps. In one embodiment, PDA is first oven-dried to a target moisture content of ≤10%. In some embodiments, PDA is oven-dried to a target moisture content of 1-10%. In some embodiments, PDA is oven-dried to a target moisture content of 1-5%. In some embodiments, PDA is oven-dried to a target moisture content of 2-3%.

In another embodiment, PDA is then granulated with excipients and a binder is incorporated. In one embodiment, PDA is granulated with microcrystalline cellulose and Starch 1500. In one embodiment, PDA is granulated with microcrystalline cellulose only. In one embodiment, PDA is blended with Starch 1500 only. In some embodiments, PDA is granulated with other commonly used tableting excipients such as but not limited to mannitol, maltodextrin, lactose, or dicalcium phosphate. In one embodiment, the binder PVP K40 is incorporated. In some embodiments, PDA is granulated with other commonly used binders such as but not limited to PVP of other molecular weights from the K40, hydroxypropyl methylcellulose (HPMC), hydroxypropyl cellulose (HPC), methylcellulose, polyethylene glycol. In some embodiments, this granulation is dried to a moisture content of ≤10%. In some embodiments, this granulation is dried to a moisture content of 3-4%. In some embodiments, water is used. In some embodiments where it is preferable to use an organic solvent during granulation instead of water, the organic solvent is used in the same amounts as specified herein for moisture content. In some embodiments, the organic solvent is methanol. In some embodiments, the organic solvent is ethanol. In some embodiments, the organic solvent is isopropyl alcohol. The organic solvents are used in situations where one or more of the components used during granulation is very soluble in water, such that use of water during granulation would dissolve the component. In such a situation, an organic solvent can be used during granulation instead of water to maintain the ingredient in a solid state.

In another embodiment, the granulation is performed using a mortar and pestle. In some embodiments, the granulation is performed using a high shear granulator. In some embodiments, the granulation is performed using a low shear granulator. In some embodiments, the granulation is performed using fluid bed granulator. In some embodiments, the resulting powder is passed through a sieve, which can have a pore size (opening size) between 10 μm and 500 μm, such as between 100 μm and 500 μm, between 200 μm and 500 μm, between 300 μm and 500 μm, between 400 μm and 500 μm, or between 400 μm and 450 μm. In some embodiments, the resulting powder is passed through a 425 μm sieve. In some embodiments, the resulting powder is blended with the remaining SYNT components, including dopamine, calcium peroxide, Tris base and acid, sodium starch glycolate, magnesium stearate, and colloidal silicon dioxide. If organic solvents were used, the organic solvents are removed by evaporation either after milling or after sieving, such that residual organic solvent is at or below the limits specified in the International Council for Harmonisation of Technical Requirements For Pharmaceuticals For Human Use—ICH Harmonised Guideline—Impurities: Guideline For Residual Solvents. The limits for maximum residual amounts of organic solvent permitted are: methanol, permissible daily exposure of 30 mg, or 3000 ppm if no more than 10 g of material are administered daily; ethanol, permissible daily exposure of 50 mg or 5000 ppm if no more than 10 g of material are administered daily; isopropyl alcohol, permissible daily exposure of 50 mg or 5000 ppm if no more than 10 g of material are administered daily.

In some embodiments, the final blend is compressed into tablets at a compaction pressure from about 500 PSI to about 5,000 PSI, such as at about 500 PSI, about 1,000 PSI, about 1,500 PSI, about 2,000 PSI, about 2,500 PSI, about 3,000 PSI, about 3,500 PSI, about 4,000 PSI, about 4,500 PSI, about 5,000 PSI, or in a range between any two of those values, such as about 1,000 PSI to about 5,000 PSI, about 2,000 PSI to about 5,000 PSI, about 3,000 PSI to about 5,000 PSI or about 4,000 PSI to about 5,000 PSI. In some embodiments, the final blend is compressed into tablets at a compaction pressure of about 4,500 PSI. In some embodiments, the final blend is compressed into tablets at a compaction pressure from about 3 megapascal to about 36 megapascal, at about 12 megapascal to about 36 megapascal, at about 3 megapascal, about 6 megapascal, about 9 megapascal, about 12 megapascal, about 15 megapascal, about 18 megapascal, about 21 megapascal, about 24 megapascal, about 27 megapascal, about 30 megapascal, about 33 megapascal, about 36 megapascal, or in a range between any two of those values, such as about 15 megapascal to about 36 megapascal, about 18 megapascal to about 36 megapascal, about 21 megapascal to about 36 megapascal, about 24 megapascal to about 36 megapascal, about 27 megapascal to about 36 megapascal, about 30 megapascal to about 36 megapascal, or about 30 megapascal to about 33 megapascal. In some embodiments, the final blend is compressed into tablets at a compaction pressure of about 31 megapascal.

In some embodiments, all of the SYNT components are used in the wet granulation process. In some embodiments, PDA and an excipient or binder are used in the wet granulation process, and the remaining SYNT components are blended in after the wet granulation process.

### Dry Granulation

Dry granulation can be used instead of wet granulation when desired. FIG. 24B outlines the basic steps. In one embodiment, PDA is first oven-dried to a target moisture content of ≤10%. In some embodiments, PDA is oven-dried to a target moisture content of 1-10%. In some embodiments, PDA is oven-dried to a target moisture content of 1-5%. In some embodiments, PDA is oven-dried to a target moisture content of 2-3%.

In another embodiment, PDA is then granulated with excipients and a binder is incorporated. In one embodiment, PDA is granulated with microcrystalline cellulose and Starch 1500. In one embodiment, PDA is granulated with microcrystalline cellulose only. In one embodiment, PDA is granulated with Starch 1500 only. In some embodiments, PDA is granulated with other commonly used tableting excipients such as but not limited to mannitol, maltodextrin, lactose, or dicalcium phosphate. In one embodiment, the binder PVP K40 is incorporated. In some embodiments, PDA is granulated with other commonly used binders such as but not limited to PVP of other molecular weights from the K40, hydroxypropyl methylcellulose (HPMC), hydroxypropyl cellulose (HPC), methylcellulose, polyethylene glycol.

In another embodiment, the granulation is performed using a slugging process. In some embodiments, the granulation would be performed by a roller compaction process. In some embodiments, the resulting powder is milled or passed through a sieve, which can have a pore size (opening size) between 10 μm and 500 μm, such as between 100 μm and 500 μm, between 200 μm and 500 μm, between 300 μm and 500 μm, between 400 μm and 500 μm, or between 400 μm and 450 μm. In some embodiments, the resulting powder is milled or passed through a 425 μm sieve. In some embodiments, the resulting powder is blended with the remaining SYNT components, including dopamine, calcium peroxide, Tris base and acid, sodium starch glycolate, magnesium stearate, and colloidal silicon dioxide.

In some embodiments, the final blend is compressed into tablets at a compaction pressure from about 500 PSI to about 5,000 PSI, such as at about 500 PSI, about 1,000 PSI, about 1,500 PSI, about 2,000 PSI, about 2,500 PSI, about 3,000 PSI, about 3,500 PSI, about 4,000 PSI, about 4,500 PSI, about 5,000 PSI, or in a range between any two of those values, such as about 1,000 PSI to about 5,000 PSI, about 2,000 PSI to about 5,000 PSI, about 3,000 PSI to about 5,000 PSI or about 4,000 PSI to about 5,000 PSI. In some embodiments, the final blend is compressed into tablets at a compaction pressure of about 4,500 PSI. In some embodiments, the final blend is compressed into tablets at a compaction pressure from about 3 megapascal to about 36 megapascal, at about 12 megapascal to about 36 megapascal, at about 3 megapascal, about 6 megapascal, about 9 megapascal, about 12 megapascal, about 15 megapascal, about 18 megapascal, about 21 megapascal, about 24 megapascal, about 27 megapascal, about 30 megapascal, about 33 megapascal, about 36 megapascal, or in a range between any two of those values, such as about 15 megapascal to about 36 megapascal, about 18 megapascal to about 36 megapascal, about 21 megapascal to about 36 megapascal, about 24 megapascal to about 36 megapascal, about 27 megapascal to about 36 megapascal, about 30 megapascal to about 36 megapascal, or about 30 megapascal to about 33 megapascal. In some embodiments, the final blend is compressed into tablets at a compaction pressure of about 31 megapascal.

### Preparation of Compressed Tablets of SYNT

Compressed solid tablets can be prepared using standard tableting equipment and procedures. The powdered starting ingredients or granulations can be blended with ribbon blenders, vertical blenders, or tumble blenders. In one embodiment, a V-type powder mixer can be used. For compression of powders, a tablet press machine can be used. For example, the Globe Pharma Tablet Press, Model MTCM, can be used, which permits compression of powder samples into tablets using hydraulic pressure applied with a manual hydraulic pump. A variety of toolings, punches, and dies are available for use, such as B tooling with a 0.330″×0.750″ capsule-shaped die. In one embodiment, the die is tapered. In some embodiments, coated tablet tooling is used, that is, the surfaces of the die and the punches that come into contact with the tablet ingredients are coated. In some embodiments, the tooling is coated with chromium nitride (CrN). In some embodiments, the tooling is coated with titanium nitride (TiN). In some embodiments, the tooling is coated with diamond-like carbon (DLC). Coated tablet tooling can also reduce the need for granulation, although optimally granulation of the powders is still performed. In some embodiments, the coated tablet tooling is used with a tapered die and increased curvature at tablet edges. Examples of tooling that can be used include, but are not limited to, round tooling of about 8 mm in diameter or modified oval tooling of about 10 mm in width and about 20 mm in length. In some embodiments, the tapered die reduces sticking during compression. In some embodiments, the increased curvature at tablet edges prevents erosion. In some embodiments, the increased curvature at tablet edges ensures uniform tablet shape.

The desired dry powders are blended, and placed in the die of the tablet press. Pressure is applied via upper and lower punches to form the compressed tablet. Pressure can be applied at about 500 PSI to about 5,000 PSI, such as at about 500 PSI, about 1,000 PSI, about 1,500 PSI, about 2,000 PSI, about 2,500 PSI, about 3,000 PSI, about 3,500 PSI, about 4,000 PSI, about 4,500 PSI, about 5,000 PSI, or in a range between any two of those values. Ranges for applied pressure can be between about 1,000 PSI to about 5,000 PSI, about 2,000 PSI to about 5,000 PSI, about 3,000 PSI to about 5,000 PSI or about 4,000 PSI to about 5,000 PSI. The applied pressure can be about 4,500 PSI. Pressure can be applied at about 3 megapascal to about 36 megapascal, at about 12 megapascal to about 36 megapascal, at about 3 megapascal, about 6 megapascal, about 9 megapascal, about 12 megapascal, about 15 megapascal, about 18 megapascal, about 21 megapascal, about 24 megapascal, about 27 megapascal, about 30 megapascal, about 33 megapascal, about 36 megapascal, or in a range between any two of those values such as about 15 megapascal to about 36 megapascal, about 18 megapascal to about 36 megapascal, about 21 megapascal to about 36 megapascal, about 24 megapascal to about 36 megapascal, about 27 megapascal to about 36 megapascal, about 30 megapascal to about 36 megapascal, or about 30 megapascal to about 33 megapascal. In some embodiments, the final blend is compressed into tablets at a compaction pressure of about 31 megapascal. For conversion purposes, 1 pound per square inch (PSI) is about 6,895 pascal, and 10,000 pascal is about 1,450 PSI.

The hardness of the resulting compressed solid tablet can be about 100 newtons, about 150 newtons, between about 100 newtons to about 400 newtons, or between about 150 newtons to about 400 newtons. Hardness can be measured according to the procedure described in the United States Pharmacopeia, Chapter 1217.

The friability of the compressed solid tablet should be 1% or less. Friability can be measured according to the procedure described in the United States Pharmacopeia, Chapter 1216.

SYNT tablets can have a total weight of 500 mg to 2000 mg, or from 250 mg to 1600 mg. Total SYNT tablet weight can be about 250 mg, 300 mg, 400 mg, 500 mg, 750 mg, 1000 mg, 1250 mg, 1500 mg, 1600 mg, 1750 mg, or 2000 mg, or an amount between any of those two values, such as from 250 mg to 500 mg, from 500 mg to 1000 mg, from 1000 mg to 1500 mg, from 1500 mg to 2000 mg, from 750 mg to 1250 mg, or from 1200 mg to 1600 mg.

### Tablet Coating for SYNT

The solid formulation may comprise a coating for SYNT. In some embodiments, the solid formulation comprises one or more coatings for SYNT.

The coating may be a tablet subcoating. In some embodiments, the tablet subcoating may be polyvinyl alcohol or hydroxypropyl methylcellulose. In some embodiments, a polyvinyl alcohol coating with polyethylene glycol plasticizer can be used, such as Opadry® II Clear 85F19250 (comprising polyvinyl alcohol, polyethylene glycol, talc and polysorbate 80) available from Colorcon (Opadry® is a registered trademark of BSPI Holdings, LLC). In some embodiments, the subcoating coating may add up to a 5% weight gain to the core tablet.

The coating may be a tablet enteric coating. In some embodiments, the tablet enteric coating may be polymethacrylates, hydroxypropyl methylcellulose phthalate, hydroxypropyl methylcellulose acetate succinate, or cellulose acetate phthalate. In some embodiments, the enteric coating may add up to a 12% weight gain to the core tablet. In some embodiments, the tablet coating or the tablet enteric coating can be coated on top of a tablet subcoating. In some embodiments, the tablet coating or the tablet enteric coating can be used without a tablet subcoating on the tablet.

In some embodiments, where delivery of SYNT to the colon is desired, polymers that can be used to target the colon include natural polymers such as chitosan, guar gum, pectin, or dextran, synthetic pH-dependent polymers such as Eudragit S100/L100, hydroxypropyl methyl cellulose phthalate, hydroxypropyl methyl cellulose acetate succinate, cellulose acetate phthalate, and time-dependent polymers such as ethylcellulose. Different variants of these polymers are used for delivery to the small intestine versus delivery to the colon. For example, for hydroxypropyl methyl cellulose acetate succinate, the higher the ratio of acetyl to succinyl groups, the higher the pH under which dissolution will occur, which can direct dissolution closer to the colon. Thus, for HPMCAS, the acetyl group to succinyl group ratio will determine where the coating and the remainder of the tablet will dissolve in the gastrointestinal tract.

### Methods of Forming a Polymer in a Gastrointestinal Tract of a Subject

Also described herein are methods of forming a polymer in a gastrointestinal tract of a subject using the solid dosage forms or formulations, and in particular compressed tablet dosage forms as disclosed herein. In some embodiments, the compressed solid tablet comprises a polymer precursor; an oxygen source; and a disintegrant. In some embodiments, the polymer precursor and the oxygen source contact a catalyst endogenous to the subject and the catalyst polymerizes the polymer precursor to form the polymer. In some embodiments, the polymer adheres to the lining of the gastrointestinal tract. In some embodiments, orally administering one or more compressed solid tablets delivers about 800 to about 5,000 mg of polymer precursor to the gastrointestinal tract. In some embodiments, orally administering one or more compressed solid tablets comprises orally administering eight tablets or fewer to the subject. In some embodiments, orally administering one or more compressed solid tablets comprises orally administering five tablets or fewer to the subject.

In some embodiments, the polymer coating is formed in the gastrointestinal tract. In some embodiments, the polymer coating is formed in the small intestine. In some embodiments, the polymer coating is formed in the duodenum. In some embodiments, the polymer coating is formed in the jejunum. In some embodiments, the polymer coating is formed in the ileum.

### Methods of Decreasing Permeability Across a Small Intestine of a Subject

Also described herein are methods of forming a polymer in a gastrointestinal tract of a subject using the solid dosage forms or formulations, and in particular compressed tablet dosage forms as disclosed herein. In some embodiments, the compressed solid tablet comprises a polymer precursor; an oxygen source; and a disintegrant. In some embodiments, the methods disclosed herein comprise orally administering one or more compressed solid tablets disclosed herein, wherein the polymer precursor and the oxygen source contact a catalyst endogenous to the small intestine of the subject and the catalyst polymerizes the polymer precursor to form a polymer. In some embodiments, the decreasing permeability comprises decreasing permeability of a sugar. In some embodiments, the sugar is glucose. In some embodiments, a decrease in permeability of at least about 5% in the subject is achieved within a period of time post administration, compared to a subject who did not receive the compressed solid tablet. In some embodiments, a decrease in permeability of at least about 25% in the subject is achieved within a period of time post administration, compared to a subject who did not receive the compressed solid tablet. In some embodiments, a decrease in permeability of at least about 50% in the subject is achieved within a period of time post administration. In some embodiments, a decrease in permeability of at least about 65% in the subject is achieved within a period of time post administration. A decrease in permeability can be measured by methods known to one in the art.

In some embodiments, the period of time is between about 30 minutes to about 240 minutes post administration. In some embodiments, the decrease in permeability in the subject is achieved within about 60 minutes. In some embodiments, the decrease in permeability in the subject is achieved within about 90 minutes. In some embodiments, the decrease in permeability in the subject is achieved within about 120 minutes. In some embodiments, the decrease in permeability in the subject is achieved within about 180 minutes. In some embodiments, the decrease in permeability in the subject is achieved within about 240 minutes.

### Methods for Treating an Illness, Disease, Disorder, or Condition

Also described are methods of treating an illness, disease, disorder, or condition in a subject, comprising administering using the solid dosage forms or formulations, and in particular compressed tablet dosage forms as disclosed herein. In some embodiments, the methods of treating an illness, disease or condition in a subject comprises administering one or more compressed solid tablet dosage forms as disclosed herein. In some embodiments, the disease or disorder is a metabolic disorder, a digestive disorder, metabolic syndrome, bleeding, an ulcer, a bowel obstruction, diabetes, obesity, hyperinsulinemia, Crohn's disease, ulcerative colitis, malabsorption, inflammatory bowel disease, irritable bowel syndrome, lactose intolerance, or Celiac disease. In some embodiments, the disease is obesity, hyperinsulinemia, or diabetes. In some embodiments, the disease is obesity. In some embodiments, the disease is hyperinsulinemia. In some embodiments, the disease is diabetes.

### EXEMPLARY EMBODIMENTS

The invention is further described by the following exemplary embodiments.

Embodiment 1. A compressed solid tablet for oral administration for forming a polymer in situ in a subject, comprising:
a polymer precursor;
an oxygen source; and
a disintegrant.

Embodiment 2. The compressed solid tablet of embodiment 1, wherein the maximum dimension of the tablet is about 20 mm or less.

Embodiment 3. The compressed solid tablet of embodiment 1, wherein the volume of the tablet is about 1.5 cubic centimeters or less.

Embodiment 4. The compressed solid tablet of any one of embodiments 1-3, wherein the polymer precursor comprises polydopamine.

Embodiment 5. The compressed solid tablet of any one of embodiments 1-3, wherein the polymer precursor comprises polydopamine and dopamine monomers.

Embodiment 6. The compressed solid tablet of any one of embodiments 1-5, wherein the oxygen source comprises urea hydrogen peroxide, hydrogen peroxide, or calcium peroxide.

Embodiment 7. The compressed solid tablet of any one of embodiments 1-5, wherein the oxygen source comprises calcium peroxide.

Embodiment 8. The compressed solid tablet of any one of embodiments 1-7, wherein the disintegrant is selected from the group consisting of croscarmellose sodium, crospovidone, and sodium starch glycolate.

Embodiment 9. The compressed solid tablet of any one of embodiments 1-7, wherein the disintegrant comprises sodium starch glycolate.

Embodiment 10. The compressed solid tablet of any one of embodiments 1-9, wherein the weight of the tablet is between about 500 mg to 2,000 mg.

Embodiment 11. The compressed solid tablet of any one of embodiments 1-10, wherein the polymer precursor comprises between about 20% to about 70% of the tablet by weight.

Embodiment 12. The compressed solid tablet of any one of embodiments 1-10, wherein the polymer precursor comprises polydopamine or polydopamine and dopamine, and the polydopamine is present in an amount between about 200 mg to about 600 mg of the tablet.

Embodiment 13. The compressed solid tablet of any one of embodiments 1-12, wherein the oxygen source comprises between about 1% to about 20% of the tablet by weight.

Embodiment 14. The compressed solid tablet of any one of embodiments 1-13, wherein the disintegrant comprises between about 1% to about 20% of the tablet by weight.

Embodiment 15. The compressed solid tablet of any one of embodiments 1-13, wherein the disintegrant comprises between about 2% to about 10% of the tablet by weight.

Embodiment 16. The compressed solid tablet of any one of embodiments 4-14, wherein the polydopamine has an average particle size of about 1200 microns or less.

Embodiment 17. The compressed solid tablet of any one of embodiments 1-16, wherein the tablet comprises a coating.

Embodiment 18. The compressed solid tablet of embodiment 17, wherein the polymer coating comprises an enteric coating.

Embodiment 19. The compressed solid tablet of any one of embodiments 1-18, wherein the tablet further comprises a buffer.

Embodiment 20. The compressed solid tablet of embodiment 19, wherein the buffer comprises Tris base, Tris acid, or a combination thereof.

Embodiment 21. The compressed solid tablet of any one of embodiments 1-20, wherein the tablet further comprises a filler.

Embodiment 22. The compressed solid tablet of embodiment 21, wherein the filler comprises microcrystalline cellulose, dicalcium phosphate, lactose, starch, or mannitol.

Embodiment 23. The compressed solid tablet of embodiment 21, wherein the filler comprises microcrystalline cellulose.

Embodiment 24. The compressed solid tablet of any one of embodiments 1-23, wherein the tablet further comprises a lubricant.

Embodiment 25. The compressed solid tablet of embodiment 23, wherein the lubricant comprises magnesium stearate, stearic acid, or sodium stearyl fumarate.

Embodiment 26. The compressed solid tablet of embodiment 23, wherein the lubricant comprises magnesium stearate.

Embodiment 27. The compressed solid tablet of any one of embodiments 1-26, wherein the tablet further comprises a glidant.

Embodiment 28. The compressed solid tablet of embodiment 27, wherein the glidant comprises silicon dioxide, talc, or corn starch.

Embodiment 29. The compressed solid tablet of embodiment 27, wherein the glidant comprises silicon dioxide.

Embodiment 30. The compressed solid tablet of embodiment 27, wherein the glidant comprises colloidal silicon dioxide.

Embodiment 31. The compressed solid tablet of any one of embodiments 1-30, wherein the tablet further comprises a binder.

Embodiment 32. The compressed solid tablet of embodiment 31, wherein the binder comprises dicalcium phosphate or Starch 1500 partially pregelatinized maize starch.

Embodiment 33. The compressed solid tablet of embodiment 31, wherein the binder comprises Starch 1500 partially pregelatinized maize starch.

Embodiment 34. The compressed solid tablet of any one of embodiments 21-33, wherein the filler comprises about 20% to about 60% by weight of the tablet.

Embodiment 35. The compressed solid tablet of any one of embodiments 31-34, wherein the amount of binder comprises about 1% to about 20% by weight of the tablet.

Embodiment 36. The compressed solid tablet of any one of embodiments 1-35, further comprising at least one permeation enhancer that enhances permeation of one or more active pharmaceutical ingredients.

Embodiment 37. The compressed solid tablet of embodiment 36, wherein the tablet comprises at least two permeation enhancers.

Embodiment 38. The compressed solid tablet of embodiment 36, wherein the tablet comprises two permeation enhancers.

Embodiment 39. The compressed solid tablet of any one of embodiments 36-38, wherein the permeation enhancers comprise a) a bile salt or bile acid, and b) an ammonium salt or a carbonate salt.

Embodiment 40. The compressed solid tablet of embodiment 39, wherein the bile salt or bile acid comprises glycocholic acid, sodium glycocholate, or a combination thereof.

Embodiment 41. The compressed solid tablet of embodiment 39 or embodiment 40, wherein the ammonium salt or carbonate salt comprises ammonium carbonate or ammonium bicarbonate.

Embodiment 42. The compressed solid tablet of embodiment 1 or embodiment 2, further comprising one or more active pharmaceutical ingredients.

Embodiment 43. The compressed solid tablet of embodiment 42, wherein the active pharmaceutical ingredient comprises a macromolecule having a molecular weight between about 1 kD and about 160 kD.

Embodiment 44. The compressed solid tablet of embodiment 43, wherein the macromolecule comprises a polypeptide.

Embodiment 45. The compressed solid tablet of embodiment 43, wherein the macromolecule comprises a polynucleotide.

Embodiment 46. The compressed solid tablet of embodiment 42, wherein the active pharmaceutical ingredient comprises a small molecule having a molecular weight of 1 kD or less.

Embodiment 47. The compressed solid tablet of any one of embodiments 1-46, wherein the tablet has a hardness of at least about 100 newtons.

Embodiment 48. The compressed solid tablet of any one of embodiments 1-46, wherein the tablet has a hardness of about 100 newtons to about 400 newtons.

Embodiment 49. The compressed solid tablet of embodiment 47 or embodiment 48, wherein the hardness is measured according to United States Pharmacopeia Chapter 1217.

Embodiment 50. The compressed solid tablet of any one of embodiments 1-49, wherein the tablet has a friability of 1% or less.

Embodiment 51. The compressed solid tablet of embodiment 50, wherein the friability is measured according to United States Pharmacopeia Chapter 1216.

Embodiment 52. A method of forming a polymer in a gastrointestinal tract of a subject, comprising orally administering one or more compressed solid tablets of any one of embodiments 1-51, wherein the polymer precursor and the oxygen source contact a catalyst endogenous to the subject and the catalyst polymerizes the polymer precursor to form the polymer.

Embodiment 53. The method of embodiment 52, wherein the polymer adheres to the lining of the gastrointestinal tract.

Embodiment 54. The method of embodiment 52 or embodiment 53, wherein orally administering one or more compressed solid tablets delivers about 800 to about 5,000 mg of polymer precursor to the gastrointestinal tract.

Embodiment 55. The method of any one of embodiments 52-54, wherein orally administering one or more compressed solid tablets comprises orally administering eight tablets or fewer to the subject.

Embodiment 56. The method of any one of embodiments 52-54, wherein the orally administering one or more compressed solid tablets comprises orally administering five tablets or fewer to the subject.

Embodiment 57. The method of any one of embodiments 52-56, wherein the polymer coating is formed in the small intestine.

Embodiment 58. The method of embodiment 57, wherein the polymer coating is formed in the duodenum.

Embodiment 59. The method of embodiment 57, wherein the polymer coating is formed in the jejunum.

Embodiment 60. The method of embodiment 57, wherein the polymer coating is formed in the ileum.

Embodiment 61. A method of decreasing permeability across a small intestine of a subject, comprising orally administering one or more compressed solid tablets of any one of embodiments 1-51, wherein the polymer precursor and the oxygen source contact a catalyst endogenous to the small intestine of the subject and the catalyst polymerizes the polymer precursor to form a polymer.

Embodiment 62. The method of embodiment 61, wherein the decreasing permeability comprises decreasing permeability of a sugar.

Embodiment 63. The method of embodiment 61 or embodiment 62, wherein a decrease in permeability of at least about 5% in the subject is achieved within a period of time post administration, compared to a subject who did not receive the compressed solid tablet.

Embodiment 64. The method of any one of embodiments 61-63, wherein a decrease in permeability of at least about 25% in the subject is achieved within a period of time post administration, compared to a subject who did not receive the compressed solid tablet.

Embodiment 65. The method of embodiment 64, wherein a decrease in permeability of at least about 50% in the subject is achieved within a period of time post administration.

Embodiment 66. The method of embodiment 64, wherein a decrease in permeability of at least about 65% in the subject is achieved within a period of time post administration.

Embodiment 67. The method of any one of embodiments 63-66, wherein the period of time is between about 30 minutes to about 240 minutes post administration.

Embodiment 68. The method of embodiment 67, wherein the decrease in permeability in the subject is achieved within about 60 minutes.

Embodiment 69. The method of embodiment 67, wherein the decrease in permeability in the subject is achieved within about 90 minutes.

Embodiment 70. The method of embodiment 67, wherein the decrease in permeability in the subject is achieved within about 120 minutes.

Embodiment 71. The method of embodiment 67, wherein the decrease in permeability in the subject is achieved within about 180 minutes.

Embodiment 72. The method of embodiment 67, wherein the decrease in permeability in the subject is achieved within about 240 minutes.

Embodiment 73. A method of delivering a therapeutically effective amount of an active agent to a subject, comprising administering one or more compressed solid tablets of any one of embodiments 1-51 to the subject.

Embodiment 74. A method of treating an illness, disease or condition in a subject, comprising administering one or more compressed solid tablets of any one of embodiments 1-51 to the subject.

Embodiment 75. The method of embodiment 74, wherein the disease or disorder is a metabolic disorder, a digestive disorder, metabolic syndrome, bleeding, an ulcer, a bowel obstruction, diabetes, obesity, hyperinsulinemia, Crohn's disease, ulcerative colitis, malabsorption, inflammatory bowel disease, irritable bowel syndrome, lactose intolerance, or Celiac disease.

Embodiment 76. The method of embodiment 74, wherein the disease is obesity, hyperinsulinemia, or diabetes.

Embodiment 77. A method of formulating the compressed solid tablet of any one of embodiments 1-51, wherein the compressed solid tablet is formulated by:
combining the polymer precursor, oxygen source, and disintegrant in a die, and
applying a force of at least about 12 megapascal to the combined polymer precursor, oxygen source, and disintegrant.

Embodiment 78. A method of formulating the compressed solid tablet of any one of embodiments 1-51, wherein the compressed solid tablet is formulated by:
combining the polymer precursor, oxygen source, disintegrant, and any additional components in a die, and
applying a force of at least about 12 megapascal to the combined polymer precursor, oxygen source, and disintegrant.

Embodiment 79. The method of embodiment 77 or embodiment 78, wherein the force is between about 12 megapascal to about 36 megapascal.

Embodiment 80. The method of any one of embodiments 77-79, wherein the compressed solid tablet has a hardness of at least about 100 newtons.

Embodiment 81. The method of any one of embodiments 77-79, wherein the compressed solid tablet has a hardness of about 100 newtons to about 400 newtons.

Embodiment 82. The method of embodiment 80 or embodiment 81, wherein the hardness is measured according to United States Pharmacopeia Chapter 1217.

### EXAMPLES

The disclosure is further illustrated by the following non-limiting examples.

### Example 1

### Preparation of Solid Dosage Tablets Via Direct Compression

1000 mg tablets comprising Active Pharmaceutical Ingredients (APIs) and excipients are prepared as follows. The APIs for the compressed tablet formulation comprise polydopamine, dopamine, Tris base, Tris acid, and calcium peroxide. The excipients for the compressed tablet formulation comprise microcrystalline cellulose (Avicel 102), sodium starch glycolate, dicalcium phosphate, magnesium stearate, and Aerosil 200 Pharma (colloidal silicon dioxide).

The components are weighed out in the following proportions:

| Component | Percentage |
|---|---|
| PDA | 48.80% |
| Dopa | 19.12% |
| Tris base | 8.45% |
| Tris acid | 4.37% |
| Calcium peroxide | 2.75% |
| MCC (Avicel 102) | 6.70% |
| Sodium starch glycolate | 5.00% |
| Dicalcium phosphate | 3.30% |
| Magnesium stearate | 1.00% |
| Aerosil 200 Pharma | 0.50% |
| (colloidal silicon dioxide) | |

The components were individually passed through an 850 μm sieve, except for the magnesium stearate and Aerosil 200. The sieved material was collected in a clean, labeled container. The sieved powders (APIs and excipients, except magnesium stearate and Aerosil 200 Pharma) were added to the powder blender (e.g., a V-blender). The powders are mixed for 10-15 minutes to achieve uniformity. The Aerosil 200 Pharma (colloidal silicon dioxide) was then added. The powders were blended for an additional 3-5 minutes. The magnesium stearate was added to the blend. The powders were blended for no more than 2-3 minutes. 1010 mg of powder was loaded into a die for the MTCM Globe Pharma Tablet Press (using B tooling with a 0.330″×0.750″ capsule-shaped die). Manual compression was applied at 4500 PSI (approximately 31 megapascals). The resulting tablets was visually inspected for integrity and uniformity.

The resulting tablet weight should be: 1000±5% mg, with the following specifications: hardness greater than 150 N; friability: <1% weight loss; appearance: smooth, unchipped, uniform in color.

The table below illustrates possible options to be included for tablet formulation.

| | Dicalcium phosphate, | 20-60% total |
|---|---|---|
| | Microcrystalline | (fillers can be |
| | cellulose, Lactose, | optionally used |
| Filler | Starch, Mannitol | in combination) |
| Disintegrant | Sodium starch glycolate, | 2-10% |
| | Croscarmellose Sodium, | |
| | Crospovidone | |
| Binders | Polyvinylpyrrolidone, | ≤5% |
| | hydroxypropyl methylcellulose | |
| Lubricant | Magnesium stearate, stearic acid, | 0.25-2% |
| | sodium stearyl fumarate | |
| Glidant | Colloidal silicon dioxide, | 0.1-2% |
| | talc, corn starch | |
| Tablet Sub | Polyvinyl alcohol, hydroxypropyl | up to 5% wt gain |
| Coating | methylcellulose | to core tablet |
| Tablet | Polymethacrylates, hydroxypropyl | up to 12% wt gain |
| Enteric | methylcellulose phthalate, | to core tablet |
| Coating | hydroxypropyl methylcellulose | |
| | acetate succinate, cellulose acetate | |
| | phthalate | |

### Example 2

### Administration of SYNT Formulations to Pigs

Once the pigs were fully anesthetized, an endoscope was inserted orally to visualize and locate the duodenum. The duodenum was identified by advancing the scope past the pyloric sphincter and recognizing its characteristic mucosal pattern and curvature. With the duodenum visualized, an overtube was carefully advanced over the endoscope and positioned within the duodenum. Once the overtube was in place, the endoscope was withdrawn, leaving the overtube in position. The tablet(s) were then delivered directly through the overtube into the duodenum. No water or PBS was flushed after tablet placement, as disintegration was expected to occur upon contact with the duodenal environment.

As the endoscope was withdrawn (after tablet or capsule delivery), visual confirmation was made to ensure that the tablets had reached the duodenum. If any tablets were observed in the stomach, this was documented. The tablets were allowed to remain in situ for 45 minutes to enable full deployment and GSEL formation. During this period, no additional substances were administered.

After the 45-minute deployment window, the endoscope was reinserted into the duodenum to confirm visual evidence of tablet deployment (i.e., tablet dissolution). Once deployment was verified, 1.4 g/kg (equivalent to 2.8 mL/kg) of a 50% w/v glucose solution was administered into the duodenum over 10 minutes using flexible tubing threaded through the working channel of the endoscope. Blood glucose levels were then monitored via ear prick every 15 minutes for four hours using a glucometer.

### Results

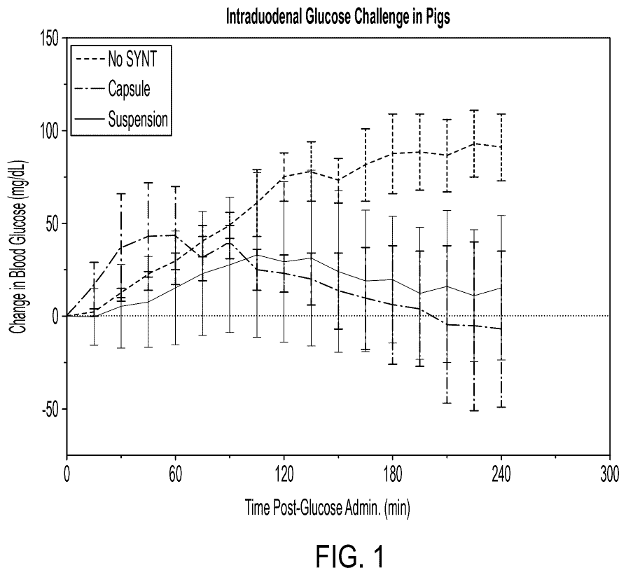

Pigs were administered a control, SYNT liquid suspension or SYNT capsules, and the change in blood glucose levels were monitored. Pigs who received SYNT (irrelevant of suspension or capsules) exhibited significantly less change in blood glucose levels compared to pigs who did not receive SYNT (FIG. 1). SYNT administered in capsule format was just as effective at suppressing changes in blood glucose compared to the liquid format. This suppression effect was durably observed over time (FIG. 1).

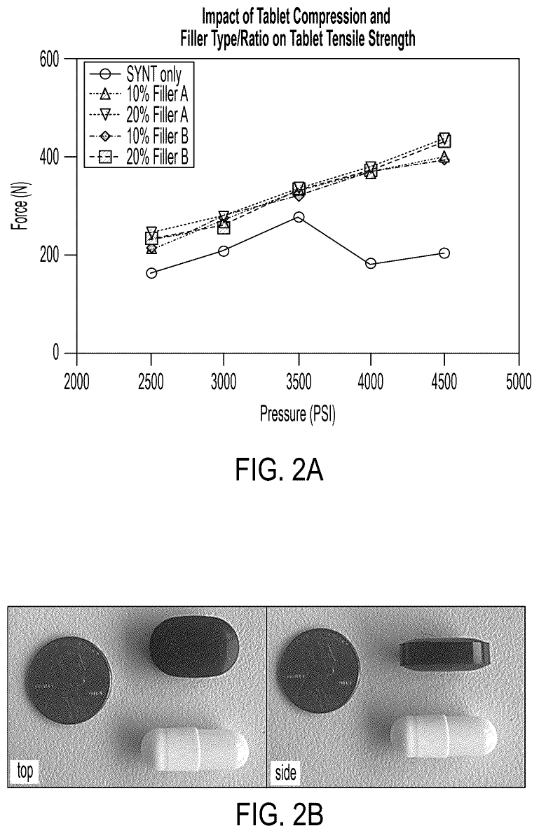

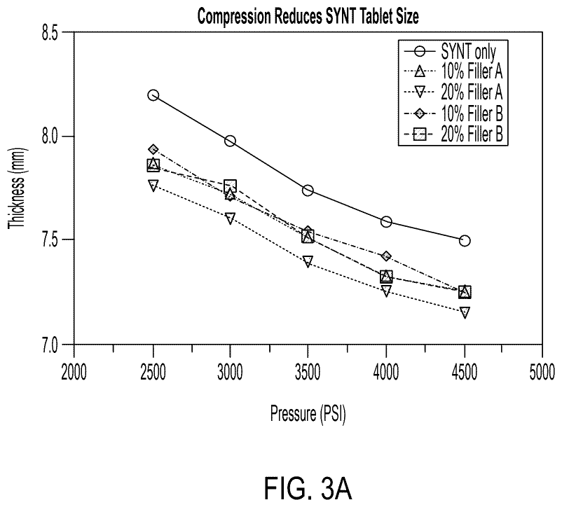

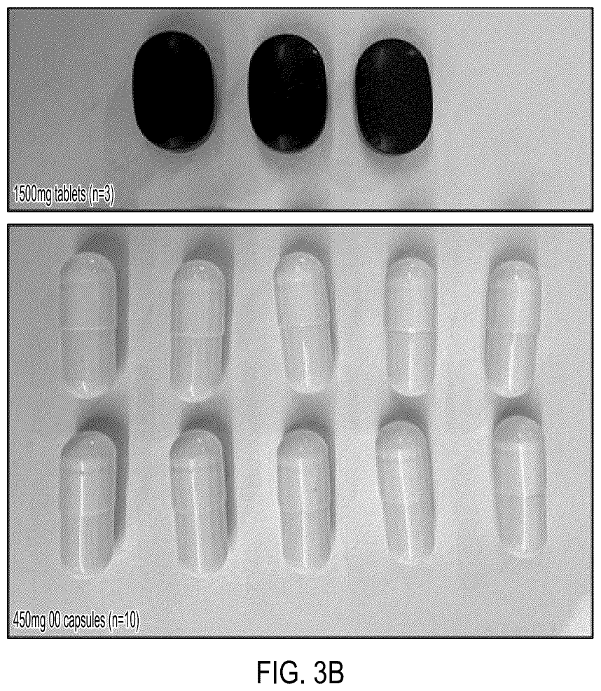

Because capsules were observed to have similar efficacy as suspension, tablets were synthesized using different fillers at various ratios to test if tablets were a viable strategy for SYNT delivery. If successful, compressed tablets could significantly reduce frequency of dosing or quantity of doses administered. In all cases, including fillers in the SYNT formulation successfully yielded tablets. Tablet tensile strength increased when the SYNT formulations including filler was compressed with higher pressure (FIG. 2A, FIG. 2B), whereas SYNT formulation when compressed without filler exhibited lower tensile strength if made using higher compression forces (such as 4000 PSI and above) (FIG. 2A). It was observed that tablet thickness decreased when higher pressure that was applied during compression (FIG. 3A). Because tensile strength increased and tablet thickness decreased, it was observed that compressed tablets were able to deliver significantly more SYNT compared to capsules (FIG. 3B). For example, the equivalent of ten capsules could be compressed into 3 tablets (FIG. 3B).

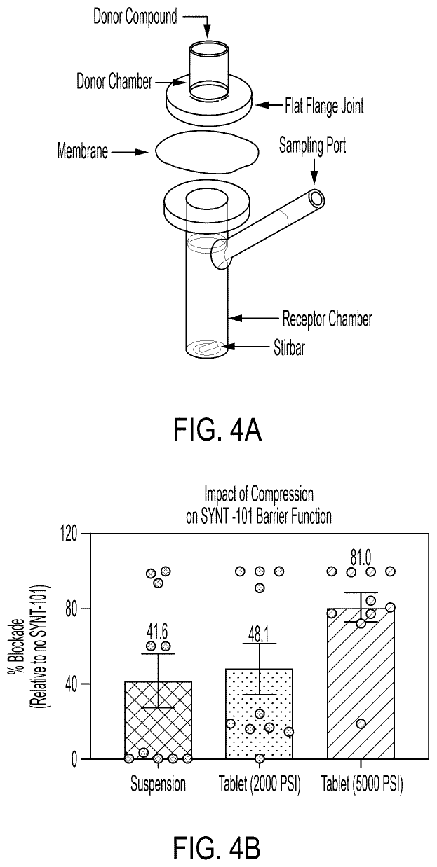

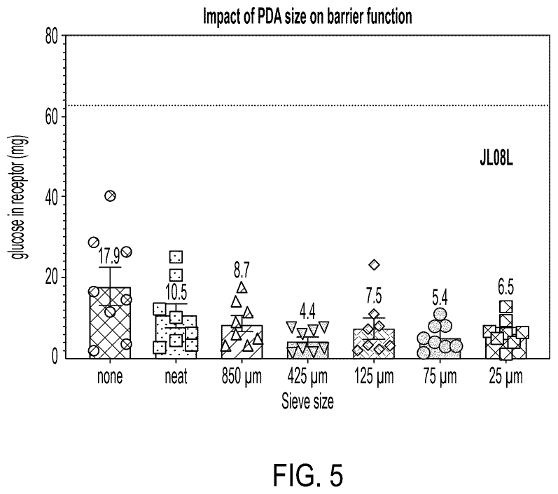

Compressed tablets were tested in vitro for efficacy in establishing barrier function (FIG. 4A). Compressed tablet subjected to dissolution or a neat suspension was added to the donor chamber, and flow-through was measured for ingredients from the donor chamber (FIG. 4A, 4B). While a tablet made with 2000 PSI exhibited similar blockade as the suspension formulation, the tablet synthesized with 5000 PSI of force exhibited an almost 2-fold enhancement in percent blockade achieved by the SYNT formulation. (For example, 81% blockade versus 41.6% percent blockade for the suspension formula) (FIG. 4B). To test if PDA particle size influenced blockade function, tablets were re-synthesized, but with PDA particles that had been sieved using different mesh sizes. It was observed that sieving to exclude PDA particles of a specified size did not significantly impact barrier function (FIG. 5, Table 3).

| Sieve size (μm) | Glucose in receptor (mg) |
|---|---|
| None | 17.9 |
| Neat (no sieving) | 10.5 |
| 850 | 6.7 |
| 425 | 4.4 |
| 125 | 7.5 |
| 75 | 5.4 |
| 25 | 6.33 |

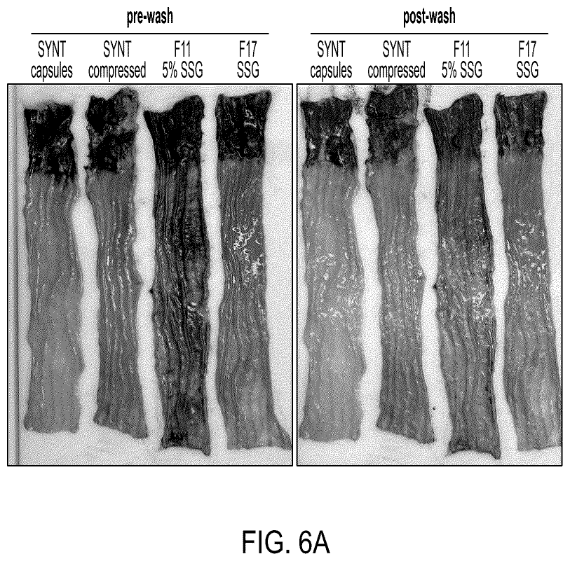

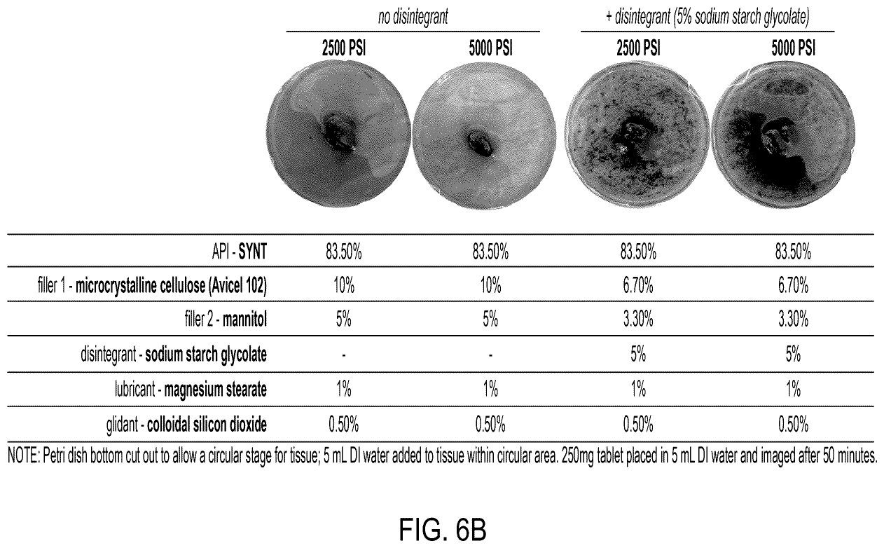

Disintegrants were investigated for inclusion into the compressed tablet to assist in tablet dissolution and release of SYNT on porcine tissue (FIG. 6A). A fresh segment of pig duodenum was flushed with phosphate-buffered saline (PBS) to remove any residual contents. A tablet was placed inside the lumen, and a syringe pump was used to perfuse fasted state simulated intestinal fluid (FaSSIF) at a flow rate of 0.7 mL/min for 45 minutes. After the perfusion period, the duodenum was cut open longitudinally, and the spread of the formulation was documented and measured. Finally, the tissue was rinsed thoroughly with 100 mL of water to assess residual retention. Porcine tissue was incubated with SYNT capsules, SYNT tablets without disintegrant, SYNT tablet with 5% sodium starch glycolate (SSG) (FIG. 6A, F11 formulation), and SYNT tablet with 2.5% SSG (FIG. 6A, F17 formulation).

Including 5% SSG permitted effective SYNT formulation release and spreading prewash, and coating post-wash compared to capsules or compressed SYNT tablets without disintegrant (FIG. 6A). Tablets compressed under different pressures were tested to evaluate if 5% SSG also enhanced dissolution. Including 5% SSG enhanced SYNT release, and including disintegrant into the SYNT tablet compressed at 5000 PSI permitted greater SYNT release from the tablet relative to the tablet compressed at 2000 PSI (FIG. 6A, 6B). This suggested that 5% SSG (or disintegrant) facilitated release of SYNT in situ. Notably, the tablets compressed at 2000 PSI and at 5000 PSI contained the same amount of SYNT and disintegrant—that is, they were the same weight, although the tablet compressed at 5000 PSI was smaller—but the 5000-PSI-compressed tablet demonstrated better coverage than the 2000-PSI-compressed tablet.

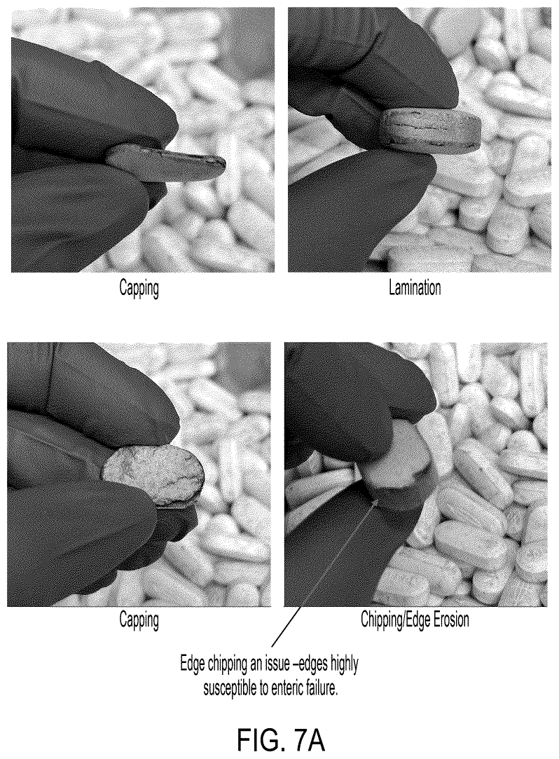

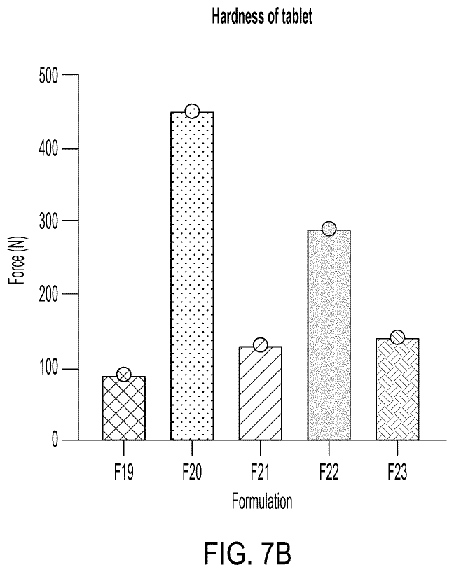

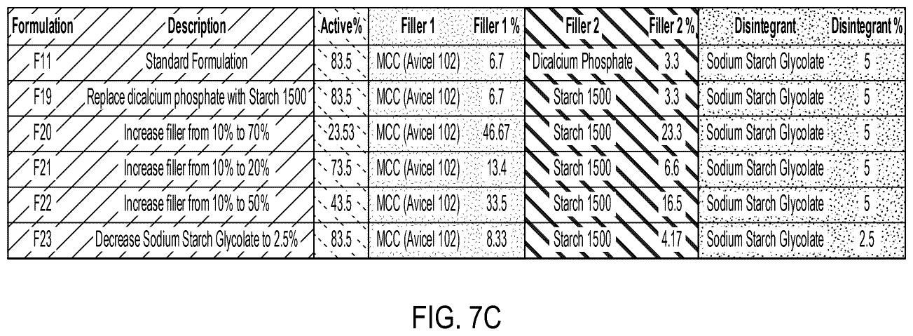

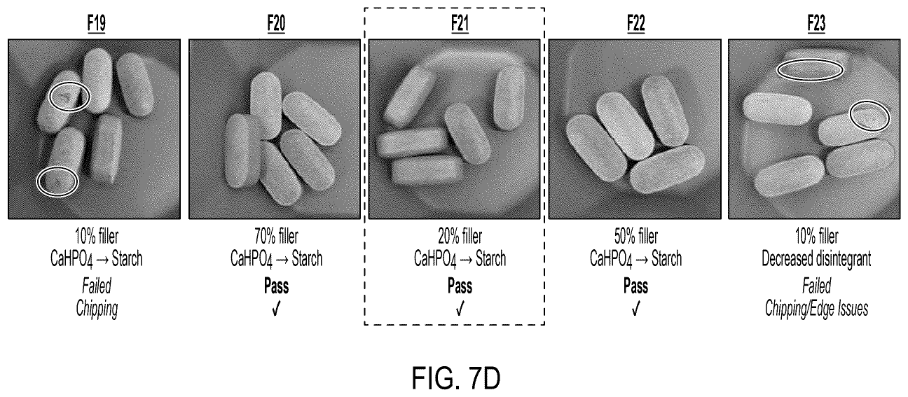

Compressed tablets can often suffer from manufacturing defects that compromise tablet efficacy such as edge chipping (which cause the edges to be susceptible to enteric failure), and excipients may be included to buffer or minimize tablet deformation (FIG. 7A, FIG. 7B, and FIG. 7C). Changing one of the fillers from dicalcium phosphate (F19) to starch 1500 (F20, F21, F22, and F23) enhanced tablet hardness (FIG. 7B, FIG. 7C). Increasing filler from 10% to 50% (F22) or to 70% (F20) significantly enhanced tablet hardness and physically enhanced the tablet to guard against physical defects such as chipping (FIG. 7D).

A compressed tablet formulation with minimal filler was selected with the goal of reducing filler and still achieving a suitable hardness, which allowed the pill burden to be lower (FIG. 7D).

### Example 3

### Evaluation of SYNT Tablets for Blockade Efficacy in Porcine Models

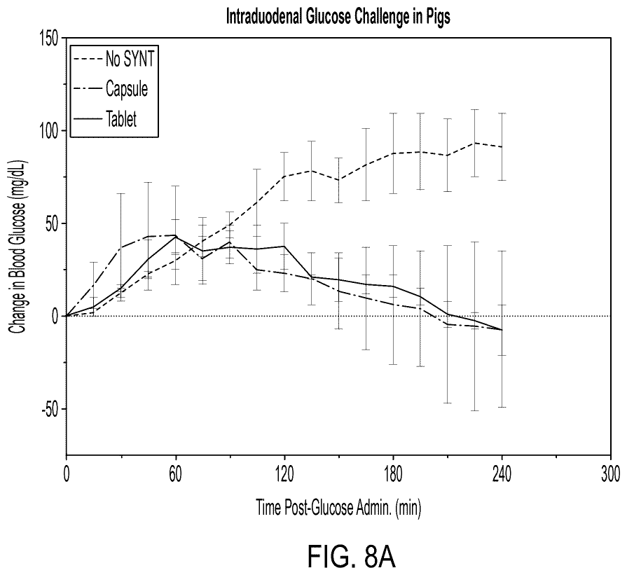

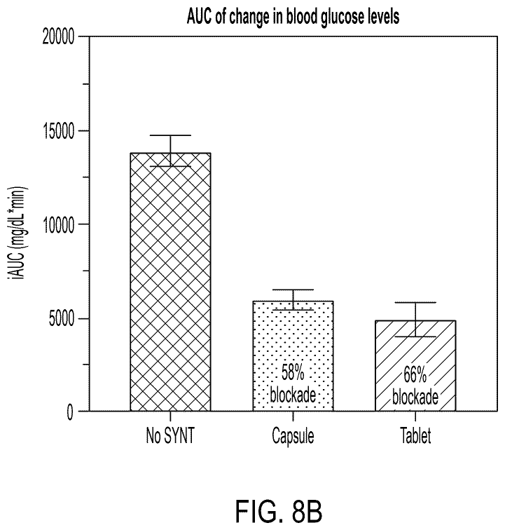

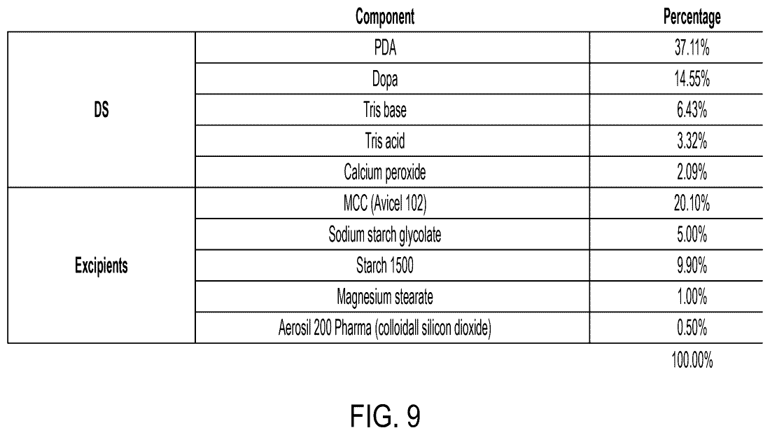

Similar to the initial experiment comparing the SYNT suspension to the capsule format to evaluate efficacy in blockade, pigs were administered a control, or SYNT capsules or SYNT tablets, and the change in blood glucose levels were monitored (FIG. 8A, FIG. 8B). A non-limiting, exemplary SYNT formulation for a compressed dosage form is shown in FIG. 9.

Pigs who received SYNT (irrelevant of tablet or capsule) exhibited significantly less change in blood glucose levels compared to pigs who did not receive SYNT (FIG. 8A). However, SYNT tablets had a significantly smaller error bars compared to SYNT capsules. SYNT administered in capsule format was just as effective, if not more effective, at suppressing changes blood glucose compared to the tablet format through blockade (FIG. 8A, 8B). This suppression effect was durably observed over time (FIG. 8A).

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

### Example 4

### Preparation of Enteric-Coated Dosage Tablets

1350 mg tablets comprising Pharmaceutical Ingredients (APIs) and excipients are prepared as follows. The APIs for the compressed tablet formulation comprise polydopamine, dopamine, Tris base, Tris acid, and calcium peroxide. The excipients for the compressed tablet formulation comprise microcrystalline cellulose (Avicel 102), sodium starch glycolate, Starch 1500, magnesium stearate, and Aerosil 200 Pharma (colloidal silicon dioxide).

The components are weighed out in the following proportions:

| Component | Percentage (w/w) |
|---|---|
| Polydopamine | 37.11 |
| Dopamine | 14.55 |
| Tris base | 6.43 |
| Tris acid | 3.32 |
| Calcium peroxide | 2.09 |
| MCC (Avicel 102) | 20.10 |
| Sodium starch glycolate | 5.00 |
| Starch 1500 | 9.90 |
| Magnesium stearate | 1.00 |
| Aerosil 200 Pharma | 0.50 |
| (colloidal silicon dioxide) | |

### Initial Blending of Active Components

The raw materials were weighed into either double polyethylene (PE)-lined containers, HDPE containers, or stainless-steel containers, as appropriate. The components were individually passed through an 850 μm sieve. The sieved material was collected in a clean, labeled container. Dopamine, polydopamine, and calcium peroxide were individually passed through a #20 mesh stainless steel screen and placed into a properly labeled double polyethylene lined container. The sieved powders were added to the powder blender (e.g., a Double Core blender). The powders were mixed for 10-15 minutes to achieve uniformity. Approximately half of the initial blend was place into a properly labeled double polyethylene lined container.

### Final Blending

Avicel 102 and Aerosil 200 (colloidal silicon dioxide) were combined, screened through a #30 mesh stainless steel screen, and placed into a properly labeled double polyethylene-lined container. Avicel 102, Starch 1500, sodium starch glycolate, and Aerosil 200 were then added to the powder blender and blended for an additional 10-15 minutes to achieve uniformity. Magnesium stearate was screened through a #30 mesh stainless steel screen. Screened magnesium stearate was added to the blender and blended for an additional 3 minutes.

### Compression

1350 mg of powder was loaded into a die for the MTCM Globe Pharma Tablet Press (using B tooling with a 0.340″×0.810″ capsule-shaped die). Manual compression was applied at 4500 PSI (approximately 31 megapascals). The resulting tablets were visually inspected for integrity and uniformity.

The resulting tablet weight should be: 1350±5% mg, with the following specifications: hardness greater than 150 N; friability: <1% weight loss; appearance: smooth, unchipped, uniform in color.

### Film Coating

The 1350 mg tablets comprising Active Pharmaceutical Ingredients (APIs) and excipients are coated as follows. The film coating for the compressed tablet formulation comprises Opadry® II—Clear 85F19250, Titanium Dioxide, and purified water.

| Component | mg/tablet |
|---|---|
| Opadry ® II-Clear 85F19250 | 40.50 |
| Titanium Dioxide | 11.95 |
| Purified Water USP | 209.79 |

Purified Water, USP was placed into a stainless-steel container. An air operated Lightnin®, propeller type mixer was placed into the Purified Water, USP. Opadry® II Clear 85F19250 (composed of polyvinyl alcohol, polyethylene glycol, talc and polysorbate 80) was added into the vortex of purified water and was mixed for a minimum of 45 minutes. The solution was allowed to stand for a minimum of 10 minutes without mixing. Mixing was resumed at a reduced speed while coating. The 12″ O'Hara coating machine was set up with the following parameters:
Inlet temperature was set to maintain an outlet temperature of 38° C. (37-50° C.)
Flow Rate: 10 ml/min (2 ml/min-12 ml/min)
Atomization Air Pressure: 40 psi (15 psi-45 psi)
Pattern Air Pressure: 25 psi (20 psi-30 psi)
Pan Speed: 8.5 RPM (5 RPM-12 RPM)
Nozzle size: 1 mm
Inlet Air: 200 CFM (180 CFM-210 CFM)

The flow rate of the pump was calibrated, and the cores were loaded into the O'Hara coating pan. Cores were pre-warmed at inlet air temperature of 41° C. (Range: 39° C.-45° C.) for about 5 minutes. Before spraying, weights of the average warmed core tablets were measured. Cores were sprayed with coating solution until the average sub-coated active tablets weight gain was achieved. After achieving the desired weight gain for the coat, tablets were dried for about 5 minutes. The sub-coated tablets were removed from the pan and placed into a clean, properly labeled, double polyethylene lined containers.

### Enteric Coating

The 1350 mg tablets comprising Active Pharmaceutical Ingredients (APIs) and excipients are coated as follows. The enteric coating for the compressed tablet formulation comprises Hypromellose Phthalate (5.0), Triethyl citrate (TEC), talc, titanium dioxide, ethanol 190 proof, and purified water.

| Component | mg/tablet |
|---|---|
| Hypromellose Phthalate (5.0) | 96.06 |
| Triethyl citrate (TEC) | 7.68 |
| Talc | 28.82 |
| Titanium dioxide | 7.68 |
| Ethanol 190 proof | 848.38 |
| Purified water, USP | 212.10 |

Sub-core tablets were weighed for enteric coating. Ethanol 190 proof was placed in a stainless-steel container equipped with a stir bar and was mixed. Hypromellose Phthalate (5.0) was added and mixed until well-dispersed. Purified water was added to the solution under stirring and mixed until dissolved. Triethyl citrate was added and mixed until dissolved. Talc and titanium dioxide were screened through a No. 60 mesh size, added separately, and mixed for a minimum of 30 minutes respectively. The dispersion was passed through a No. 60 mesh sieve prior to the coating process. The dispersion was continuously stirred during the coating process. The 12″ O'Hara coating machine was set up using the following parameters:
Inlet temperature was set to maintain an outlet temperature of 34° C. (33-38° C.)
Flow Rate: 10 ml/min (5 ml/min-15 ml/min)
Atomization Air Pressure: 40 psi (15 psi-45 psi)
Pattern Air Pressure: 20 psi (15 psi-25 psi)
Inlet Air Pressure: 200 CFM (195 CFM-205 CFM)
Pan Speed: 8.5 RPM (5 RPM-12 RPM)
Nozzle size: 1 mm

The flow rate of the pump was calibrated, and the sub-coated active tablets were loaded into the O'Hara coating pan. Cores were pre-warmed at inlet air temperature of 34° C. (Range: 33° C.-38° C.) for about 5 minutes. Before spraying, weights of the average warmed sub-coated active tablets were measured. Cores were sprayed with coating solution until the average sub-coated active tablets weight gain was achieved. After achieving the desired weight gain for the coat, tablets were dried for about 5 minutes, 10 minutes, and 20 minutes. The sub-coated tablets were removed from the pan and placed into a clean, properly labeled, double polyethylene lined containers.

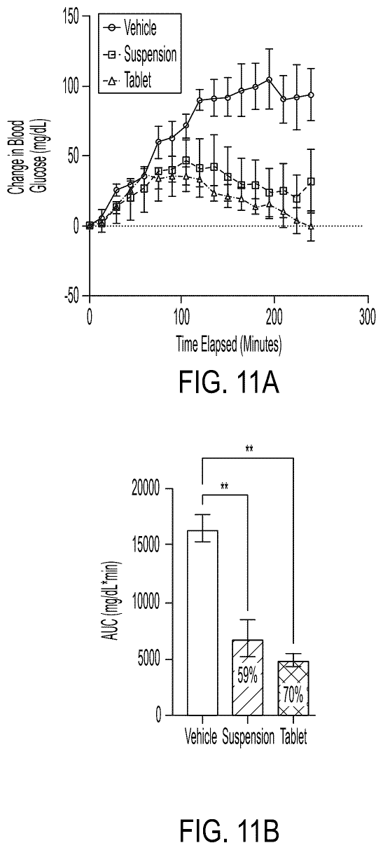

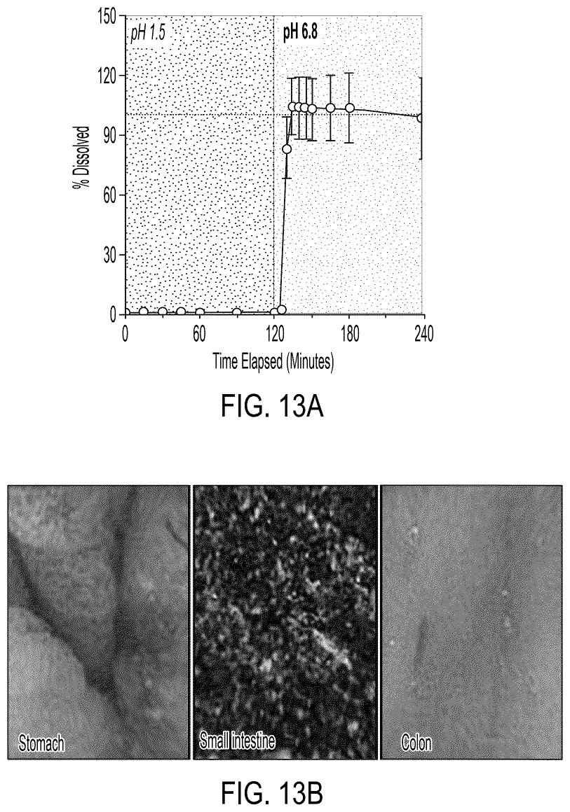

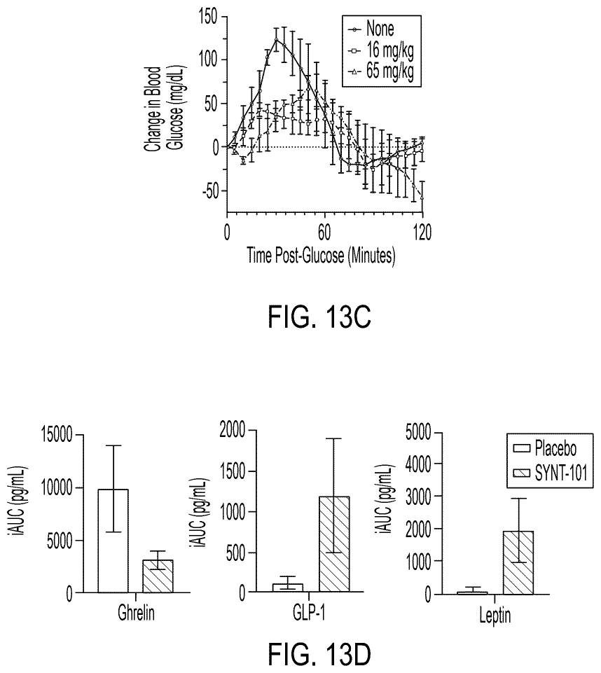

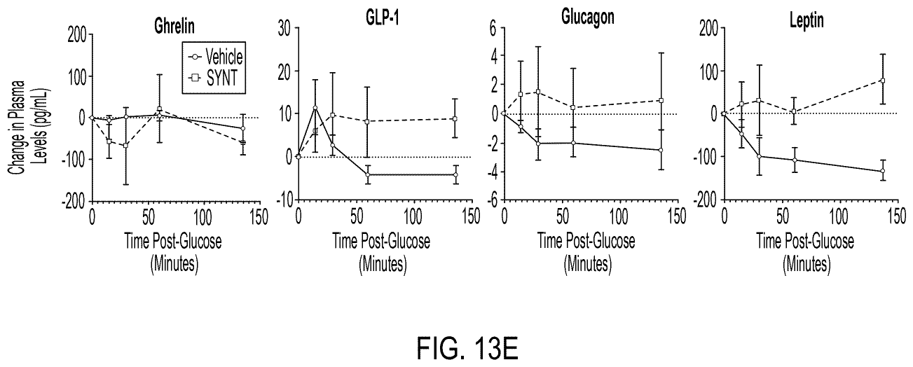

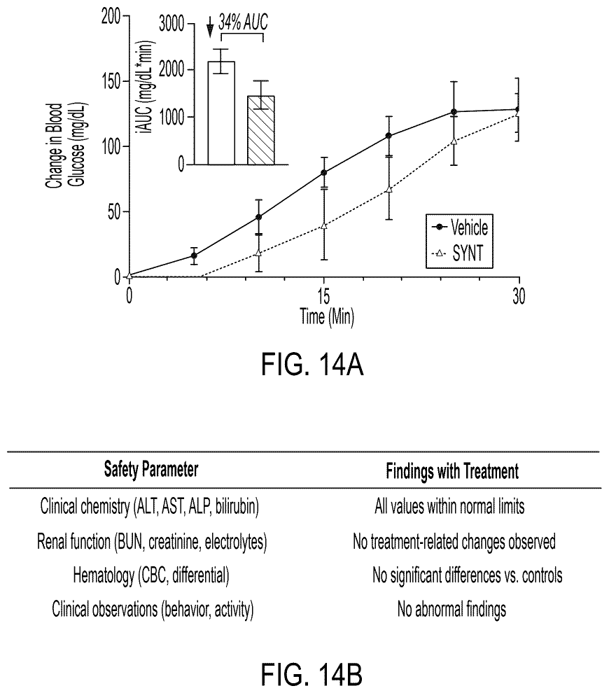

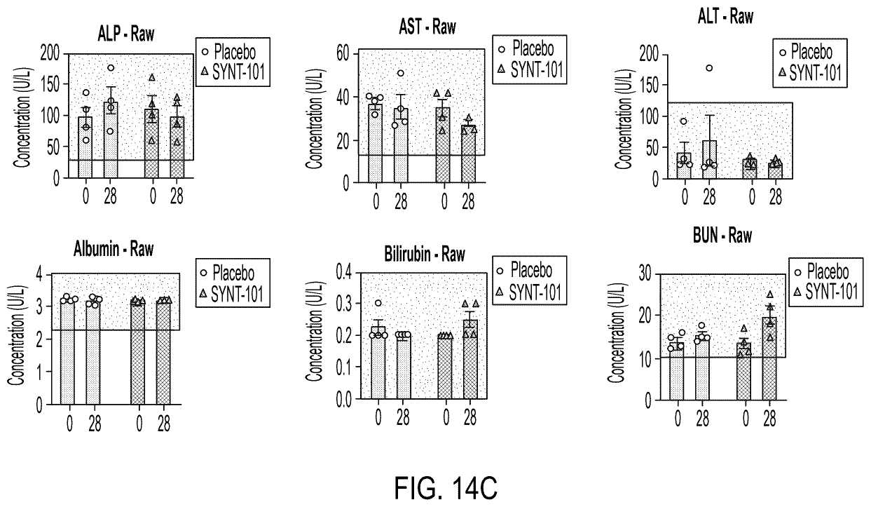

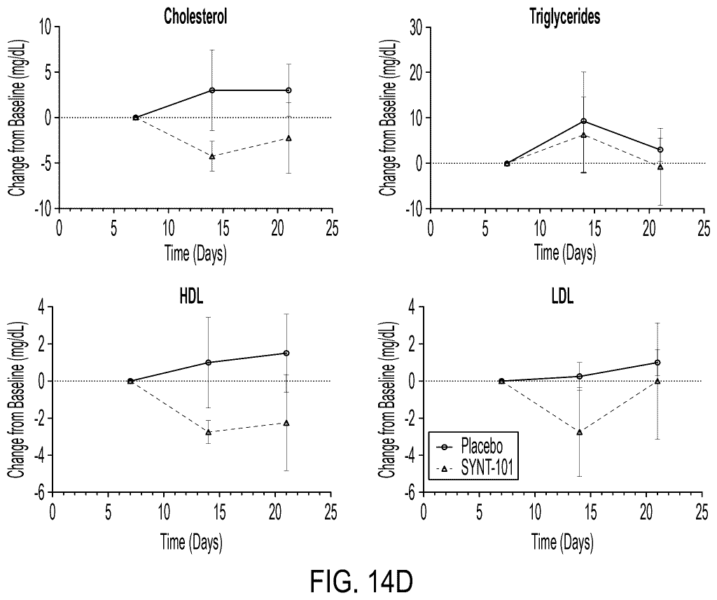

The tablets tested in FIG. 11, FIG. 13, and FIG. 14 were prepared according to this method. Tablets used in FIG. 11 were 1000 mg tablets. Tablets used in FIG. 13 and FIG. 14 were 250 mg tablets.

### Example 5

### Evaluation of Catalase Expression Levels in Tissues

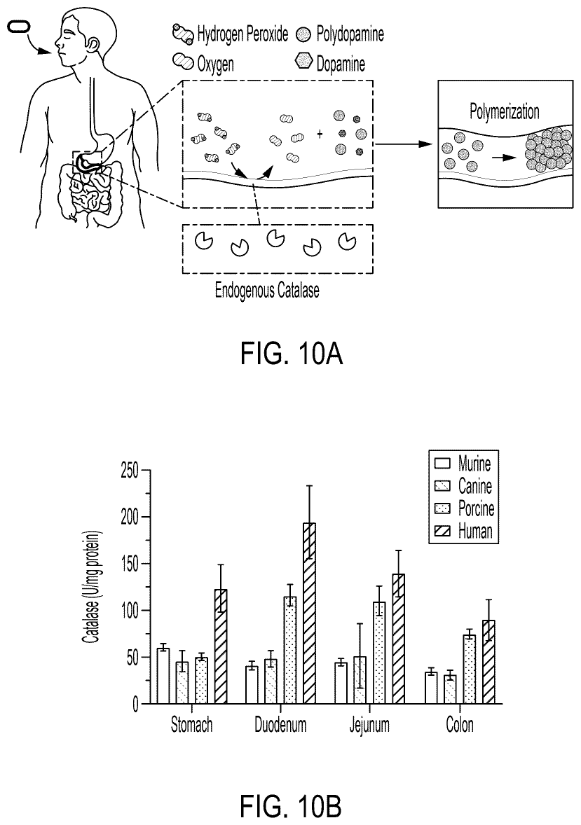

To measure catalase levels in GI tissue across species, ~1 in² of tissue was dissected, rinsed in cold 1×PBS, and dried with Kimwipes. A 0.3 g sample was weighed and minced. Tissue was homogenized on ice (1:9 w/v ratio in 1 mM EDTA, pH 7.4) using Glass Pyrex hand homogenizers for 6 minutes. Homogenates were centrifuged at 10×g for 10 minutes at 4° C. to obtain the lysate. Catalase activity was measured using the ELab Science kit (E-BC-K031-M). Lysate (20 μL) was incubated with 200 μL buffer at 37° C. for 10 minutes, followed by 20 μL substrate for 1 minute at 37° C. Then, 200 μL of the chromogenic solution and 20 μL clarification reagent were added. After 10 minutes at room temperature, the absorbance was read at 405 nm using a microplate reader. Activity (U/mL) was calculated using a standard curve. Total protein was quantified using the Pierce™ BCA Protein Assay Kit (Thermo Fisher, 23250). Lysate (9 μL) was mixed with 4 μL compatibility reagent and incubated at 37° C. for 15 minutes. Then, 260 μL working solution was added and incubated at 37° C. for 30 minutes. The plate was cooled for 5 minutes before reading absorbance at 562 nm. Protein concentration (mg/mL) was interpolated from a standard curve. Catalase activity was normalized to total protein concentration Cpr using the formula: CAT (U/mg protein)=CAT (U/mL lysate)÷Cpr (mg protein/mL lysate). See FIG. 10B.

### Example 6

### Evaluation of SYNT-101 for Duodenal-Targeted Obesity Therapy in Murine Models

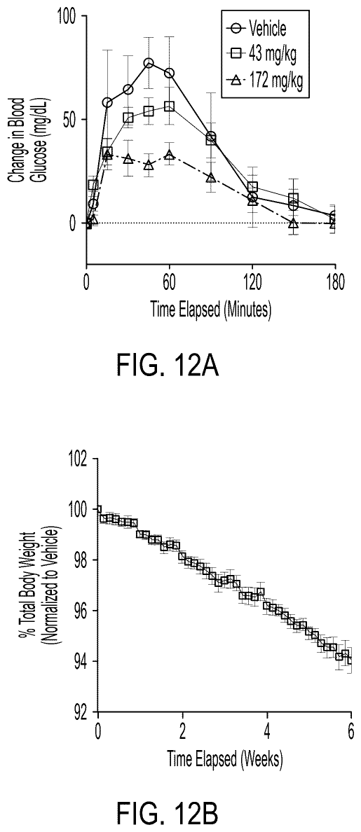

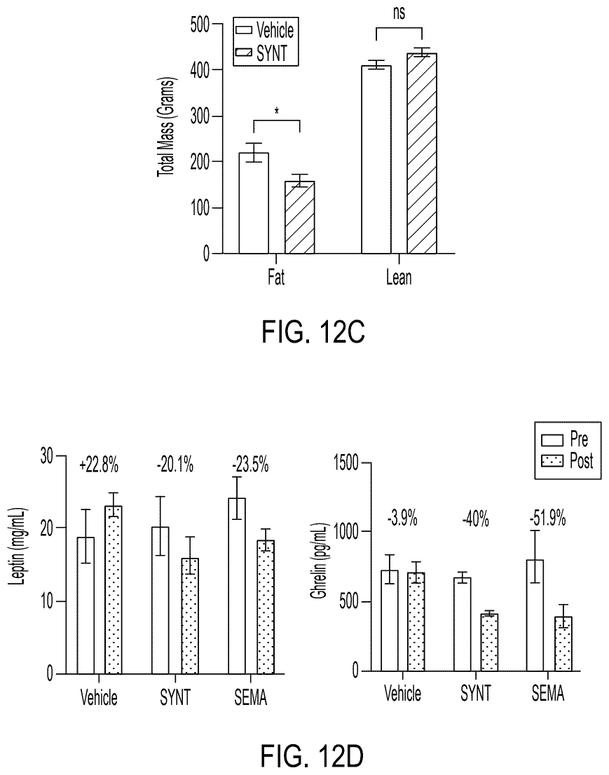

Sprague-Dawley rats (20 weeks old, n=16) were maintained on an ad libitum high-fat diet (HFD, ResearchDiets D12492) for 12 weeks prior to study initiation to promote acclimation and induce weight gain. On Study Day 0, rats were pair-stratified by weight and randomly assigned to either the treatment group (SYNT-101, n=6) or control group (vehicle, n=6). The treatment group received a daily oral gavage of SYNT-101 (2 mL/kg), while the control group received saline (2 mL/kg). Throughout the 6-week study, body weight and food consumption were monitored daily, and body composition changes, including lean and fat mass, were assessed using EchoMRI, with a focus on epididymal and mesenteric white adipose tissue (eWAT and mWAT). Plasma samples were collected at both the study initiation (Day 0) and completion (Day 42) to assess metabolic and satiety hormone profiles (FIG. 12D). At the study's conclusion, all rats were euthanized, and gastrointestinal tissue was harvested for further analysis.

SYNT-101 demonstrated visual evidence of transient deposition in the duodenum and showed a significant reduction in glucose uptake during OGTT (FIG. 12A). Clearance was visually confirmed within 12 hours of administration. The 6-week study demonstrated 1% body weight reduction each week (FIG. 12B) and an average 10% daily reduction in food intake. Importantly, the treatment preserved lean muscle mass (FIG. 12C), while achieving a 20+% reduction in fat mass, including reductions in eWAT and mWAT. Reduced adiposity with preservation of lean muscle mass was observed in treated animals. Fasted leptin and ghrelin decreased post-treatment when compared to placebo and subcutaneous semaglutide (SEMA) (FIG. 12D). Additionally, diet-induced obesity (DIO) rats demonstrated outstanding tolerability throughout the 6-week treatment period.

SYNT-101 demonstrates promising effects on weight loss, glucose metabolism, and body composition in DIO rodent models. Most notably, the treatment shows consistent, weekly weight loss, significantly reduced adiposity, while effectively preserving lean muscle mass, a critical limitation to existing weight loss interventions. These findings highlight the therapeutic potential of SYNT-101 for managing obesity and related metabolic disorders.

### Example 7

### Visualization of SYNT-101 in Canine Models

Healthy canines (~1.5 years old, n=8) were maintained on a normal diet. On Study Day 0, randomly assigned to either the treatment group (SYNT-101, n=4) or control group (vehicle, n=4). The treatment group received daily administration of SYNT-101 tablets), while the control group received placebo tablets. Throughout the study, body weight was measured. Plasma samples were collected at both the study initiation (Day 0) and completion (Day 28) to assess metabolic and satiety hormone profiles (FIG. 13D and FIG. 13E).

SYNT-101 demonstrated visual evidence of transient deposition in the duodenum (FIG. 13A). Pill-Cam imaging demonstrated SYNT-101 formation in the small intestine within two hours post-administration, with no evidence of SYNT in the stomach/colon (FIG. 13B). Results showed a significant reduction in glucose uptake during OGTT (FIG. 13C). Modulation of satiety-related hormone levels during OGTT following SYNT-101 administration at 16 mg/kg was observed (AUC calculated over four hours post-glucose administration) (FIG. 13D and FIG. 13E). Increased leptin/GLP-1 levels were observed, while ghrelin levels decreased.

Repeat-dosing of SYNT-101 tablets in canines was performed (48 mg/kg). After 28 days dosing and 24-hour washout, blood glucose levels during OGTT were reduced, particularly within the first 30 minutes post-glucose administration (n=4/group) (FIG. 14A). IDEXX blood chemistry and safety assessments showed no abnormalities relative to baseline following the 28-day dosing period (FIG. 14B, FIG. 14C, and FIG. 14D).

SYNT-101 demonstrates promising effects on glucose metabolism and body composition in canine models. Further, repeat daily dosing over 28 days was well tolerated. These findings highlight the therapeutic potential of SYNT-101 for managing obesity and related metabolic disorders.

### Example 8

### First-In-Human Evaluation of SYNT-101 for Obesity Management

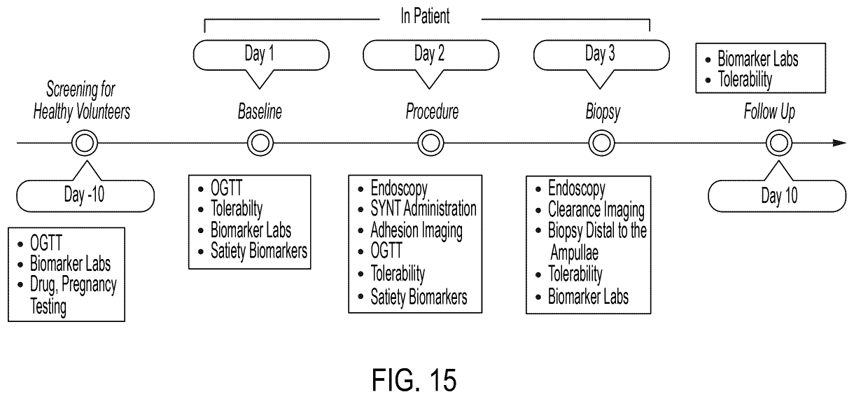

The first-in-human study evaluation of SYNT-101 for the treatment of obesity was conducted as shown in FIG. 15. The study began with screening (Day −10) and baseline assessments (Day 1), followed by SYNT administration and imaging procedures (Day 2) and biopsy (Day 3). A follow-up visit on Day 10 included biomarker and tolerability assessments. Nine healthy subjects (2 male, 7 female), aged 24 to 53 years with a BMI ranging from 19 to 29, received a single dose of SYNT-101 in a suspension formulation across multiple doses. The cohort was divided into three groups based on dosage: 25% (n=2), 50% (n=3), and 100% (n=4) of the target SYNT-101 level. Comprehensive safety assessments and oral glucose tolerance tests (OGTT) were performed to confirm SYNT-101 safety and efficacy. Endoscopic imaging was used to characterize duodenal surface coverage spatially and temporally. Plasma samples were collected to assess the impact on satiety/metabolic hormone levels (liver enzymes, leptin, ghrelin).

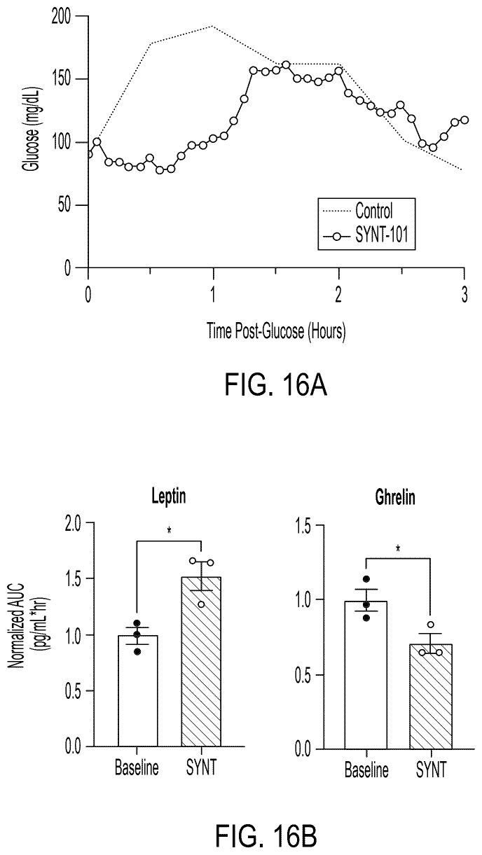

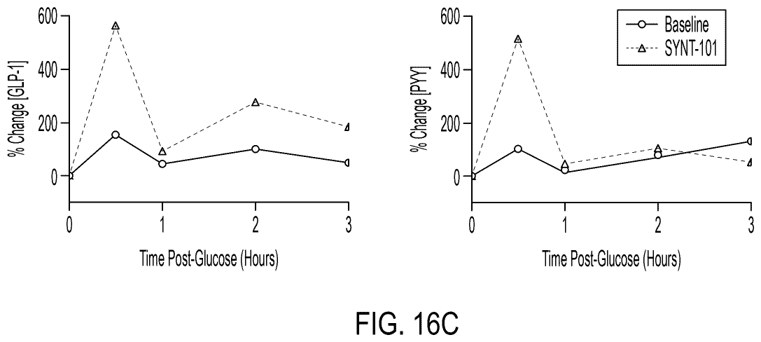

Endoscopic imaging revealed extensive SYNT-101 coverage throughout the duodenum. Safety assessments showed no adverse or serious adverse events in any treatment group (FIG. 17). Per individual, liver enzymes, including aspartate transaminase (AST), alanine transaminase (ALT), and bilirubin, remained unchanged over a ten-day period post-treatment. Gastrointestinal tolerance was excellent, with no changes observed in the Gastrointestinal Symptom Rating Scale (GSRS) and an average pain rating of 0 (n=9). Histopathological examinations conducted 24 hours post-administration showed normal duodenal mucosa, with no signs of erosion or residual SYNT-101. OGTT tests demonstrated delayed glucose absorption with SYNT-101 treatment (FIG. 16A). After SYNT-101 administration, there was an average 34.7% reduction in the area under the curve within the first 30 minutes and a 20.9% reduction within 60 minutes, suggesting nutrient redirection from the duodenum to later in the intestine. Consistent with reduced food intake in the pre-clinical in vivo models, 100% of patients tested exhibited elevated plasma leptin levels and reduced ghrelin (FIG. 16B). SYNT-101 also modulates other key metabolic hormones including GLP-1 and PYY (FIG. 16C). Furthermore, enhanced coverage and reduced variability in OGTT is shown through the development of an orally delivered solid dosage formulation (FIG. 11A and FIG. 11B).

SYNT-101 has demonstrated a strong profile of safety, tolerability, and efficacy in first-in-human studies, showing promise as a well-tolerated therapeutic option for effective weight loss solutions. Unlike many existing treatments, SYNT-101 offers a safe, tolerable, and effective oral alternative to current injectable and/or systemic therapies for weight management. SYNT-101 adheres to the intestinal mucosa and effectively redirects nutrient absorption. SYNT-101 achieved a 35% reduction in glucose AUC, demonstrating delayed nutrient absorption. Treatment induced metabolic effects, including a reduction in ghrelin and elevation of leptin, GLP-1, and PYY. OGTT response is improved when administering an oral tablet. SYNT-101 was well tolerated with no treatment-emergent adverse events, no biomarker changes, and no notable histology or pathology findings.

### Example 9

### Evaluation of PERMEATE in Porcine and Murine Models

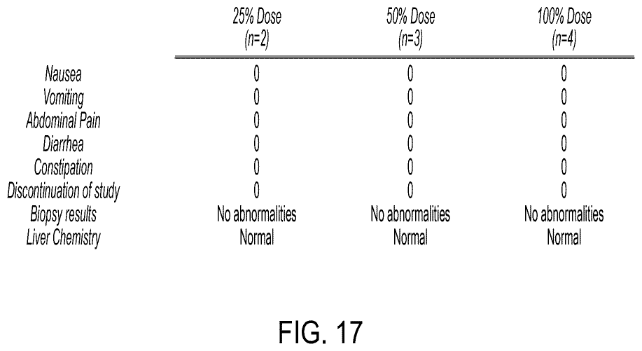

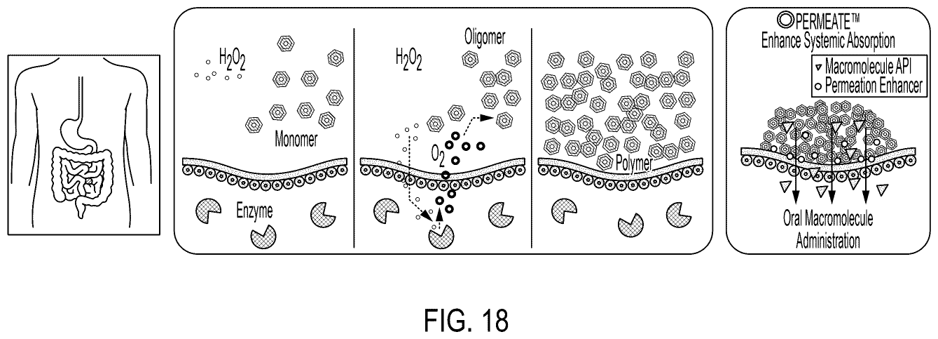

Incretin peptide therapies have revolutionized obesity and diabetes treatment, yet overcoming oral delivery challenges—such as rapid gastrointestinal transit and limited intestinal absorption—is crucial to improving patient adherence, accessibility, and real-world effectiveness. To address these barriers, the Peroral Mucosal Epithelium Absorption Enhancer (PERMEATE) method was developed, which integrates synthetic tissue lining (SYNT) platform with proprietary permeation enhancers (PEs) to improve intestinal residence time and drive higher absorption (FIG. 18). This proof-of-concept study demonstrates PERMEATE's ability to both create and significantly enhance the oral bioavailability of semaglutide (SEMA) in pig and rat models.

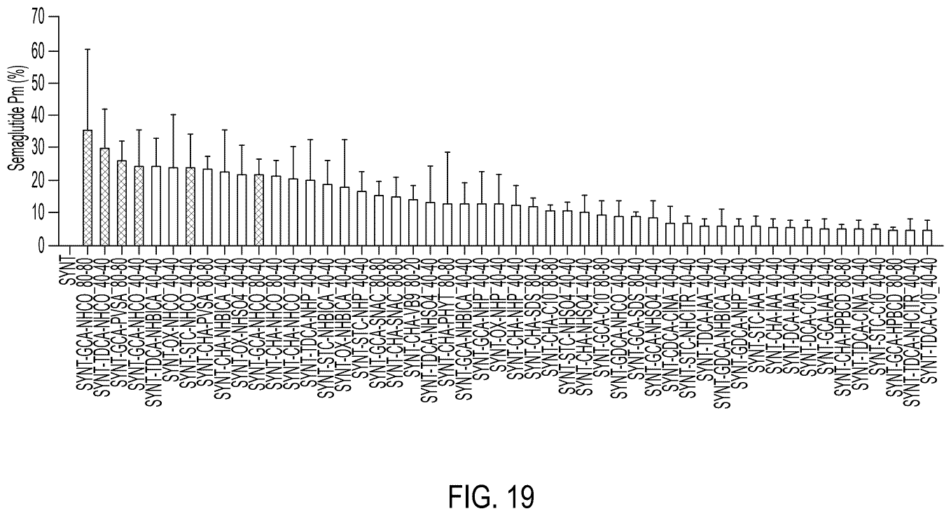

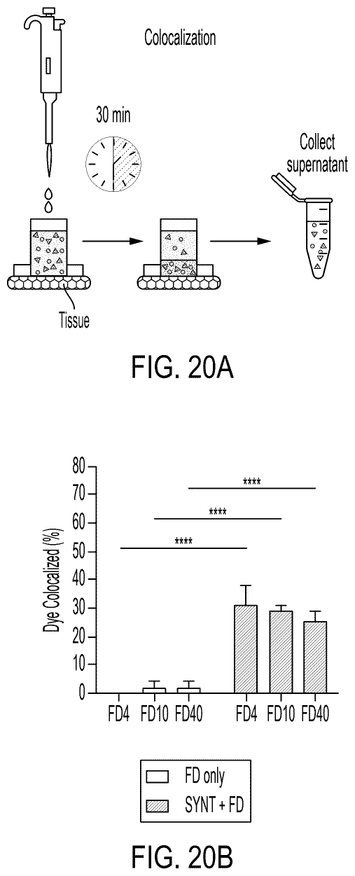

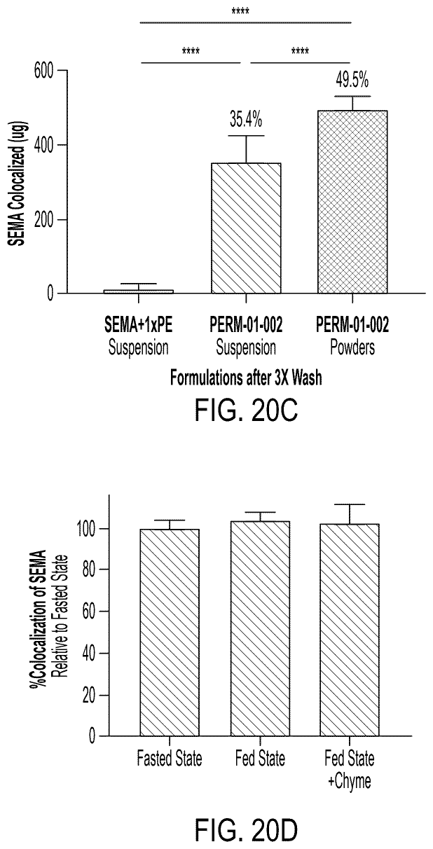

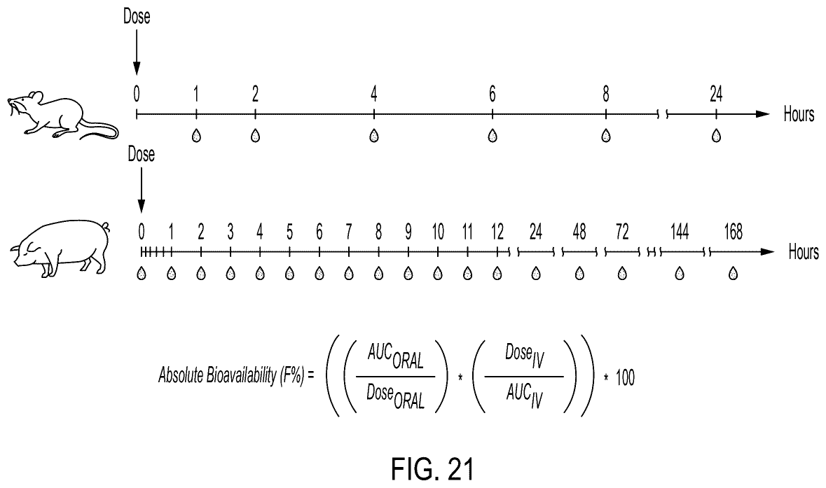

A high-throughput ex vivo screen identified single and combination permeation enhancers (PEs) with synergistic effects on SEMA permeation across porcine intestinal tissue (FIG. 19). Er vivo Franz experiments optimized formulation components and ratios to maximize colocalization of SEMA and PEs onto the intestinal tissue (FIGS. 20A-20D). Saline buffer washes were implemented to mimic a dynamic environment physiologically relevant to digestion. SYNT significantly improves entrapment of FITC-Dextran dyes (4, 10, 40 kDa) by up to 17-fold (P<0.0001) (FIG. 20B). PERMEATE retains semaglutide on duodenal tissue after 3× buffer washes. Entrapment is significantly enhanced with delivery of PERMEATE as powders, highlighting potential improvements as a solid dosage formulation (P<0.0001) (FIG. 20C). PERMEATE effectively adheres and colocalizes SEMA in fed-state tissue conditions (FIG. 20D). (Lead formulations were tested in vivo in anesthetized Yorkshire pigs, a physiologically relevant gastrointestinal model, with plasma SEMA concentrations measured via LC-MS/MS over 168 hours (FIG. 21). The area under the curve (AUC) was normalized by dose and compared to intravenous SEMA administration (n=4) to determine absolute bioavailability. To enhance replicates and rigor, formulations were also delivered via oral gavage to male rats (650-750 g, n=5-6 per group), with bioavailability assessed over 24 hours using the same methods (FIG. 21).

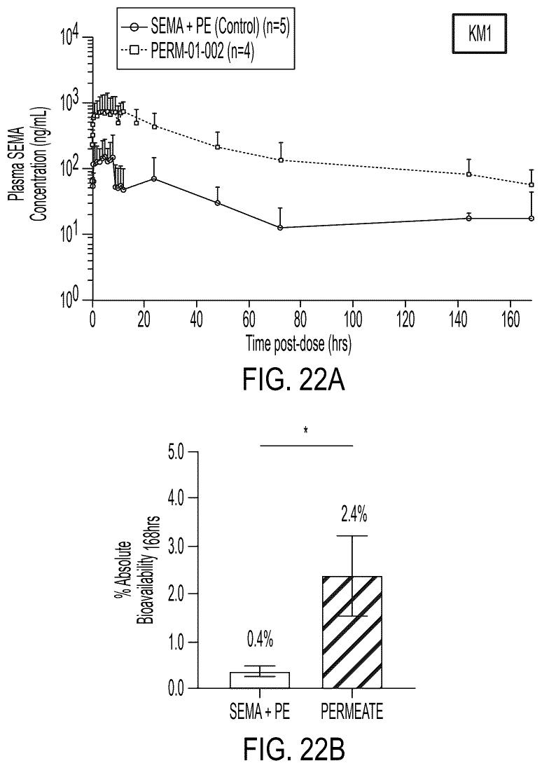

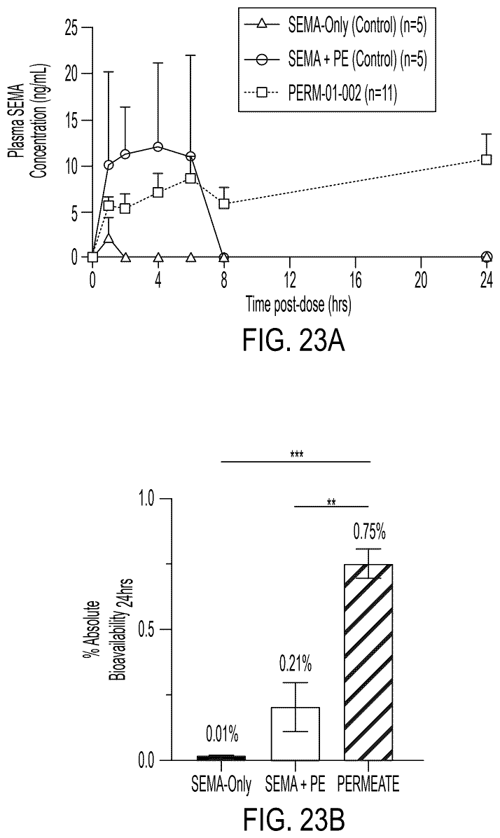

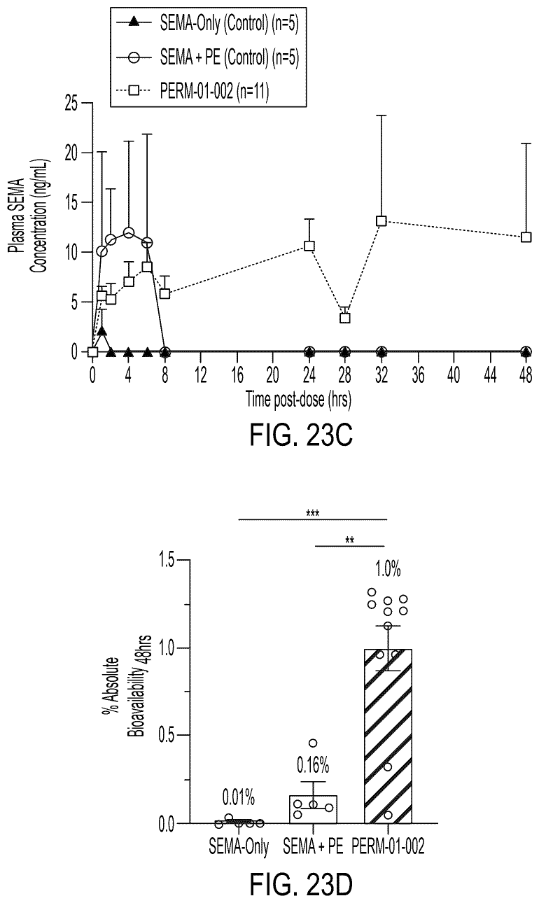

Glycocholic acid (GCA) and ammonium carbonate (NHCO) were identified as the most effective PE combination with SEMA and SYNT, achieving a 15.5-fold increase in permeation compared to SEMA control in ex vivo tests (FIG. 19). Optimized PERMEATE formulations improved colocalization of SEMA by 71.5-fold compared to a SEMA+PE control, after two washes, highlighting the ability of PERMEATE to create a localized depot and achieve prolonged residence time. In vivo, PERMEATE demonstrated a SEMA bioavailability of 2.4±1.6% in Yorkshire pigs (n=4) (FIG. 22A and FIG. 22B), representing a significant 6-fold increase over the SEMA+PE control (0.4±0.3%, n=5; p=0.0316) and SEMA-Only control (0%, n=1). Improved bioavailability compared to controls was also observed in rats, highlighting translatability across multiple mammalian models (FIG. 23A, FIG. 23B, FIG. 23C, and FIG. 23D). Absolute bioavailabilities of Semaglutide in rats demonstrate 75-fold improvement compared to SEMA-Only control (P=0.0006) and 3.6-fold improvement over SEMA+PE (P=0.0092) at 24 hours post-dose. Absolute bioavailabilities of Semaglutide in rats demonstrate 200-fold improvement compared to SEMA-Only control (P=0.0001) and 6-fold improvement over SEMA+PE (P=0.0011) at 48 hours post-dose.

The PERMEATE platform, powered by SYNT™, significantly enhances the bioavailability of semaglutide (SEMA) in preclinical models, demonstrating up to a 6-fold improvement compared to controls. PERMEATE significantly improves intestinal residence of molecules up to 40 kDa by up to 17-fold. PERMEATE retains up to 35.4% (suspension) and up to 49.5% (powders) of semaglutide after 3× buffer washes (vs. Control 0%). Additionally, PERMEATE extends semaglutide circulation up to 600% of its half-life in rats. These findings underscore the potential of PERMEATE as a transformative platform for optimizing the oral delivery of macromolecule therapeutics.

### Example 10

### Tablet Tooling for Compression of Moisture-Containing, PDA-Based SYNT-101 Formulations

Compression of SYNT-101 tablets presents significant challenges due to the tackiness of the polydopamine (PDA) polymer and the addition of moisture needed to achieve adequate tablet hardness. This often requires very high ejection forces that then lead to tablet cracking. Tablet hardness, however, is critical for maintaining mechanical integrity during handling, enteric coating, packaging, and transportation. Insufficient hardness can lead to chipping, cracking, or edge erosion, which compromises tablet shape, coating uniformity, and consistent therapeutic performance. Conventional tablet tooling and die designs are insufficient to accommodate the unique properties of PDA-containing SYNT-101 formulations, resulting in reduced yield. Prior methods for compressing moisture-sensitive or tacky polymers generally rely on standard tablet tooling and lubrication techniques. While such approaches can reduce sticking in some formulations, they are insufficient for PDA-based SYNT-101 blends, which are prone to edge erosion and mechanical defects during compression and coating. To address these challenges, optimized tooling design is developed to produce robust, hard tablets suitable for reliable enteric coating and downstream processing.

This method involves the use of coated tablet tooling with a tapered die and increased curvature at tablet edges to accommodate the tackiness and moisture content of the PDA-containing SYNT-101 formulation. The tapered die reduces sticking during compression, while the increased curvature at edges prevents erosion and ensures uniform tablet shape. This optimized tooling allows production of mechanically robust tablets suitable for subsequent enteric coating, improving coating uniformity and minimizing defects. Any coated tablet compression tooling, which includes tooling coated with chromium nitride (CrN), titanium nitride (TiN), and diamond-like carbon (DLC), can be utilized on a tablet press to achieve improved tablet robustness/hardness.

Optimized tooling enables successful compression of tacky, moisture-containing SYNT-101 tablets with minimal edge erosion and sticking. The resulting tablets have improved hardness, uniformity, and coating compatibility, allowing consistent enteric coating and enhanced mechanical integrity. Compared with conventional tooling, this optimized tooling reduces production defects and increases overall yield of high-quality coated tablets.

### Example 11

### Wet Granulation of Polydopamine to Improve SYNT Tabletability

The moisture content of polydopamine (PDA) influences tablet integrity and mechanical strength during the enteric coating process. At present, moisture is manually reintroduced to oven-dried PDA to preserve tablet robustness during coating. Incorporation of a controlled wet granulation step enables more precise regulation of PDA moisture content and improves formulation consistency and mechanical performance. However, wet granulation cannot be applied to the full SYNT blend due to the moisture sensitivity of dopamine and calcium peroxide. As such, a method was developed to granulate polydopamine with excipients, thereby reducing the need for the addition of moisture earlier in the production process.

The wet granulation process is illustrated in FIG. 24A. Polydopamine (PDA) is first oven-dried to a target moisture content of ≤10%. The PDA is then wet granulated with excipients (for example, microcrystalline cellulose and Starch 1500), with addition of water. The PDA may be granulated with only microcrystalline cellulose or with only Starch 1500. Granulation with other commonly used tableting excipients may also occur, such as with mannitol, maltodextrin, lactose, or dicalcium phosphate. A binder, such as PVP K40, is also incorporated. Other commonly used binders for tablet formulation may also be incorporated, such as PVP of other molecular weights from the K40, hydroxypropyl methylcellulose (HPMC), hydroxypropyl cellulose (HPC), methylcellulose, or polyethylene glycol. This granulation is then dried to a moisture content of ≤10%, such as 3-4%, and subsequently sieved by passing through a sieve, which may have a pore size of between 10 μm to 500 μm, and blended with the remaining SYNT components, including dopamine, calcium peroxide, Tris base and acid, sodium starch glycolate, magnesium stearate, and colloidal silicon dioxide. The final blend is compressed into tablets at a compaction pressure of up to 5,000 psi (up to 34.5 megapascals). Alternatively, the remaining SYNT components may be used in the wet granulation rather than blending them in after granulation.

This approach improves tablet integrity by enhancing interparticle bonding and blend homogeneity, resulting in increased mechanical strength and reduced friability during compression and downstream processing. Overall, this represents a robust and effective wet granulation strategy for this drug product while maintaining the stability of moisture-sensitive components.

An alternative dry granulation process is illustrated in FIG. 24B.

What is claimed is:

1. A compressed solid tablet for oral administration for forming a polymer in situ in a subject, comprising:
a polymer precursor;
an oxygen source; and
a disintegrant.

2. The compressed solid tablet of claim 1, wherein the maximum dimension of the tablet is about 20 mm or less.

3. The compressed solid tablet of claim 1, wherein the volume of the tablet is about 1.5 cubic centimeters or less.

4. The compressed solid tablet of any one of claims 1-3, wherein the polymer precursor comprises polydopamine.

5. The compressed solid tablet of any one of claims 1-3, wherein the polymer precursor comprises polydopamine and dopamine monomers.

6. The compressed solid tablet of any one of claims 1-3, wherein the polymer precursor comprises polydopamine, dopamine oligomers, and dopamine monomers.

7. The compressed solid tablet of any one of claims 1-6, wherein the oxygen source comprises a peroxide.

8. The compressed solid tablet of any one of claims 1-6, wherein the oxygen source comprises urea hydrogen peroxide, hydrogen peroxide, calcium peroxide, sodium percarbonate, or a hydrogen peroxide-polyvinylpyrrolidone complex.

9. The compressed solid tablet of any one of claims 1-6, wherein the oxygen source comprises calcium peroxide.

10. The compressed solid tablet of any one of claims 1-9, wherein the disintegrant is selected from the group consisting of croscarmellose sodium, crospovidone, and sodium starch glycolate.

11. The compressed solid tablet of any one of claims 1-9, wherein the disintegrant comprises sodium starch glycolate.

12. The compressed solid tablet of any one of claims 1-11, wherein the weight of the tablet is between about 500 mg to 2,000 mg.

13. The compressed solid tablet of any one of claims 1-11, wherein the weight of the tablet is between about 250 mg to 1,600 mg.

14. The compressed solid tablet of any one of claims 1-13, wherein the polymer precursor comprises between about 20% to about 70% of the tablet by weight.

15. The compressed solid tablet of any one of claims 1-13, wherein the polymer precursor comprises polydopamine or polydopamine and dopamine, and the polydopamine is present in an amount between about 200 mg to about 600 mg of the tablet.

16. The compressed solid tablet of any one of claims 1-15, wherein the oxygen source comprises between about 1% to about 20% of the tablet by weight.

17. The compressed solid tablet of any one of claims 1-16, wherein the disintegrant comprises between about 1% to about 20% of the tablet by weight.

18. The compressed solid tablet of any one of claims 1-16, wherein the disintegrant comprises between about 2% to about 10% of the tablet by weight.

19. The compressed solid tablet of any one of claims 4-17, wherein the polydopamine has an average particle size of about 1200 microns or less.

20. The compressed solid tablet of any one of claims 1-19, wherein the tablet comprises a coating.

21. The compressed solid tablet of any one of claims 1-19, wherein the tablet comprises a polymer coating.

22. The compressed solid tablet of claim 21, wherein the polymer coating comprises an enteric coating.

23. The compressed solid tablet of any one of claims 1-22, wherein the tablet further comprises a buffer.

24. The compressed solid tablet of claim 23, wherein the buffer comprises Tris base, Tris acid, or a combination thereof.

25. The compressed solid tablet of any one of claims 1-24, wherein the tablet further comprises a filler.

26. The compressed solid tablet of claim 25, wherein the filler comprises microcrystalline cellulose, dicalcium phosphate, lactose, starch, or mannitol.

27. The compressed solid tablet of claim 25, wherein the filler comprises microcrystalline cellulose.

28. The compressed solid tablet of any one of claims 1-27, wherein the tablet further comprises a lubricant.

29. The compressed solid tablet of claim 28, wherein the lubricant comprises magnesium stearate, stearic acid, or sodium stearyl fumarate.

30. The compressed solid tablet of claim 28, wherein the lubricant comprises magnesium stearate.

31. The compressed solid tablet of any one of claims 1-30, wherein the tablet further comprises a glidant.

32. The compressed solid tablet of claim 31, wherein the glidant comprises silicon dioxide, talc, or corn starch.

33. The compressed solid tablet of claim 31, wherein the glidant comprises silicon dioxide.

34. The compressed solid tablet of claim 31, wherein the glidant comprises colloidal silicon dioxide.

35. The compressed solid tablet of any one of claims 1-34, wherein the tablet further comprises a binder.

36. The compressed solid tablet of claim 35, wherein the binder comprises dicalcium phosphate or Starch 1500 partially pregelatinized maize starch.

37. The compressed solid tablet of claim 35, wherein the binder comprises Starch 1500 partially pregelatinized maize starch.

38. The compressed solid tablet of any one of claims 25-37, wherein the filler comprises about 20% to about 60% by weight of the tablet.

39. The compressed solid tablet of any one of claims 35-38, wherein the amount of binder comprises about 1% to about 20% by weight of the tablet.

40. The compressed solid tablet of any one of claims 1-39, further comprising at least one permeation enhancer that enhances permeation of one or more active pharmaceutical ingredients.

41. The compressed solid tablet of claim 40, wherein the tablet comprises at least two permeation enhancers.

42. The compressed solid tablet of claim 40, wherein the tablet comprises two permeation enhancers.

43. The compressed solid tablet of any one of claims 40-42, wherein the permeation enhancers comprise a) a bile salt or bile acid, and b) an ammonium salt or a carbonate salt.

44. The compressed solid tablet of claim 43, wherein the bile salt or bile acid comprises glycocholic acid, sodium glycocholate, or a combination thereof.

45. The compressed solid tablet of claim 43 or claim 44, wherein the ammonium salt or carbonate salt comprises ammonium carbonate or ammonium bicarbonate.

46. The compressed solid tablet of any one of claims 1-45, further comprising one or more active pharmaceutical ingredients.

47. The compressed solid tablet of claim 46, wherein the active pharmaceutical ingredient comprises a macromolecule having a molecular weight between about 1 kD and about 160 kD.

48. The compressed solid tablet of claim 47, wherein the macromolecule comprises a polypeptide.

49. The compressed solid tablet of claim 47, wherein the macromolecule comprises a polynucleotide.

50. The compressed solid tablet of claim 46, wherein the active pharmaceutical ingredient comprises a small molecule having a molecular weight of 1 kD or less.

51. The compressed solid tablet of any one of claims 1-50, wherein the tablet has a hardness of at least about 100 newtons.

52. The compressed solid tablet of any one of claims 1-50, wherein the tablet has a hardness of about 100 newtons to about 400 newtons.

53. The compressed solid tablet of claim 51 or claim 52, wherein the hardness is measured according to United States Pharmacopeia Chapter 1217.

54. The compressed solid tablet of any one of claims 1-53, wherein the tablet has a friability of 1% or less.

55. The compressed solid tablet of claim 54, wherein the friability is measured according to United States Pharmacopeia Chapter 1216.

56. A method of forming a polymer in a gastrointestinal tract of a subject, comprising orally administering one or more compressed solid tablets of any one of claims 1-55, wherein the polymer precursor and the oxygen source contact a catalyst endogenous to the subject and the catalyst polymerizes the polymer precursor to form the polymer.

57. The method of claim 56, wherein the polymer adheres to the lining of the gastrointestinal tract.

58. The method of claim 56 or claim 57, wherein orally administering one or more compressed solid tablets delivers about 800 to about 5,000 mg of polymer precursor to the gastrointestinal tract.

59. The method of any one of claims 56-58, wherein orally administering one or more compressed solid tablets comprises orally administering eight tablets or fewer to the subject.

60. The method of any one of claims 56-58, wherein the orally administering one or more compressed solid tablets comprises orally administering five tablets or fewer to the subject.

61. The method of any one of claims 56-60, wherein the polymer coating is formed in the small intestine.

62. The method of claim 61, wherein the polymer coating is formed in the duodenum.

63. The method of claim 61, wherein the polymer coating is formed in the jejunum.

64. The method of claim 61, wherein the polymer coating is formed in the ileum.

65. The method of any one of claims 56-60, wherein the polymer coating is formed in the large intestine.

66. A method of decreasing permeability across a small intestine of a subject, comprising orally administering one or more compressed solid tablets of any one of claims 1-55, wherein the polymer precursor and the oxygen source contact a catalyst endogenous to the small intestine of the subject and the catalyst polymerizes the polymer precursor to form a polymer.

67. The method of claim 66, wherein the decreasing permeability comprises decreasing permeability of a sugar.

68. The method of claim 66 or claim 67, wherein a decrease in permeability of at least about 5% in the subject is achieved within a period of time post administration, compared to a subject who did not receive the compressed solid tablet.

69. The method of any one of claims 66-68, wherein a decrease in permeability of at least about 25% in the subject is achieved within a period of time post administration, compared to a subject who did not receive the compressed solid tablet.

70. The method of claim 69, wherein a decrease in permeability of at least about 50% in the subject is achieved within a period of time post administration.

71. The method of claim 69, wherein a decrease in permeability of at least about 65% in the subject is achieved within a period of time post administration.

72. The method of any one of claims 68-71, wherein the period of time is between about 30 minutes to about 240 minutes post administration.

73. The method of claim 72, wherein the decrease in permeability in the subject is achieved within about 60 minutes.

74. The method of claim 72, wherein the decrease in permeability in the subject is achieved within about 90 minutes.

75. The method of claim 72, wherein the decrease in permeability in the subject is achieved within about 120 minutes.

76. The method of claim 72, wherein the decrease in permeability in the subject is achieved within about 180 minutes.

77. The method of claim 72, wherein the decrease in permeability in the subject is achieved within about 240 minutes.

78. A method of delivering a therapeutically effective amount of an active agent to a subject, comprising administering one or more compressed solid tablets of any one of claims 1-55 to the subject.

79. A method of treating an illness, disease or condition in a subject, comprising administering one or more compressed solid tablets of any one of claims 1-55 to the subject.

80. The method of claim 79, wherein the disease or disorder is a metabolic disorder, a digestive disorder, metabolic syndrome, bleeding, an ulcer, a bowel obstruction, diabetes, obesity, hyperinsulinemia, Crohn's disease, ulcerative colitis, malabsorption, inflammatory bowel disease, irritable bowel syndrome, lactose intolerance, or Celiac disease.

81. The method of claim 79, wherein the disease is obesity, hyperinsulinemia, or diabetes.

82. A method of formulating the compressed solid tablet of any one of claims 1-55, wherein the compressed solid tablet is formulated by:
combining the polymer precursor, oxygen source, and disintegrant in a die, and
applying a force of at least about 12 megapascal to the combined polymer precursor, oxygen source, and disintegrant.

83. A method of formulating the compressed solid tablet of any one of claims 1-55, wherein the compressed solid tablet is formulated by:
combining the polymer precursor, oxygen source, disintegrant, and any additional components in a die, and
applying a force of at least about 12 megapascal to the combined polymer precursor, oxygen source, disintegrant, and any additional components.

84. The method of claim 82 or claim 83, wherein the force is between about 12 megapascal to about 36 megapascal.

85. The method of any one of claims 82-84, wherein the compressed solid tablet has a hardness of at least about 100 newtons.

86. The method of any one of claims 82-85, wherein the compressed solid tablet has a hardness of about 100 newtons to about 400 newtons.

87. The method of claim 85 or claim 86, wherein the hardness is measured according to United States Pharmacopeia Chapter 1217.
